(19) United States
(12) Patent Application Publication
(10) Pub. No.: US 2025/0213779 A1
(43) Pub. Date: Jul. 3, 2025

(54) MEDICAL DEVICE HUB POSITION DETECTION SYSTEM, METHOD, AND APPARATUS

(71) Applicants: BAXTER INTERNATIONAL INC., Deerfield, IL (US); BAXTER HEALTHCARE SA, Glattpark (Opfikon) (CH)

(72) Inventors: Peter BOJAN, Grayslake, IL (US); Gnana D. PRASUNA, Andhra Pradesh (IN); Siba Prasad SAHU, Bangalore (IN); David James SCHMIDT, Gurnee, IL (US); Subbaiah PATIBANDLA, Karnataka Bangalore (IN); Erik Michael THOMAS, Gurnee, IL (US); Niranjan MANJUNATH, Bangalore (IN); Yogesh MUNAVALLI, Karnatake Bangalore (IN); Jiri SLABY, Buffalo Grove, IL (US)

(21) Appl. No.: 19/004,998

(22) Filed: Dec. 30, 2024

(30) Foreign Application Priority Data
Dec. 28, 2023 (IN) 202341089494

(51) Int. Cl. A61M 5/14; A61M 5/142; G16H 40/67

(52) U.S. Cl. CPC A61M 5/1413; A61M 5/142; G16H 40/67; A61M 2205/14; A61M 2205/3569; A61M 2205/50; A61M 2205/60

(57) ABSTRACT

A system, method, and apparatus are disclosed for determining positions of medical devices within a hub, which comprises a number of apparatuses that are linked together in a stacked configuration. A medical device hub includes a connectivity apparatus that is communicatively coupled to a medical network and/or a monitoring device. Additionally, the medical device hub includes one or more docking apparatuses. Each docking apparatus can accommodate two or more infusion pumps. The number of docking apparatuses used in the medical device hub depends on the number of infusion pumps needed for a patient treatment. The medical device hub enables multiple docking apparatuses to be stacked as needed while enabling the infusion pumps to be operated independently and removed from the medical device hub as needed, even during a treatment without interruption. Such a configuration provides a scalable, flexible, and adaptable system that aggregates infusion pumps into a relatively small footprint.

### PRIORITY CLAIM

This application claims priority to and the benefit as a non-provisional application of Indian Provisional Patent Application No. 202341089494 filed Dec. 28, 2023, the entire contents of which are hereby incorporated by reference and relied upon.

### BACKGROUND

Infusion pumps are medical devices that infuse a fluid into a patient's bloodstream or subcutaneously. Infusion therapies often use pumps to delivery nutrition in addition to certain medications such as pain medications, antibiotics, antiemetics, antifungals, antivirals, biologics, blood factors, chemotherapy drugs, corticosteroids, growth hormones, immunoglobulin replacement fluids, immunotherapy fluids, and inotropic heart medications. There are different types of infusion pumps that are optimized for delivering different types of fluids or medications. For instance, a syringe pump may be used for delivering low volumes of potent medications at relatively slow rates while large volume parenteral (“LVP”) pumps are used for delivering greater volumes of nutritional fluids. Other types of known infusion pumps include linear peristaltic pumps, patient-controlled analgesia (“PCA”) pumps, ambulatory pumps, and multi-channel pumps.

Some patient treatments may require the use of more than one infusion pump. For example, a first infusion pump may deliver saline while a second pump delivers a medication. Currently, clinicians can either connect individual pumps to a patient or use a multi-channel pump. When individual pumps are used, the pumps are often spread around a patient's hospital bed. The clinician has to move around the patient's bed to program each pump individually. In addition, the pumps consume a significant amount of space. When a multi-channel pump is used, a clinician only has to program one device and less space is used. However, multi-channel pumps generally only support one type of infusion pump, such as large volume pumps. If another pump is needed for a treatment, a clinician still has to add that infusion pump in addition to the multi-channel pump, thereby reducing the efficiencies of the multi-channel pump.

A need accordingly exists for a medical device hub that supports different types of infusion pumps and other medical devices.

### SUMMARY

An example system, method, and apparatus are disclosed for medical device position detection within a medical device hub. Unlike known multi-channel infusion pump systems with a fixed two, four, or six pumps, the medical device hub described herein is modular. In a base configuration, the medical device hub includes a single medical device (infusion pump) docking apparatus and a connectivity apparatus, which connects the hub to a hospital network or a monitoring device. The docking apparatus includes two shelves for respectively receiving infusion pumps or other medical devices. In an embodiment, the shelves are configured to interchangeably accommodate different types of infusion pumps, such as syringe pumps, LVP pumps, PCA pumps, etc. based on which type of pump is needed for a particular treatment. Further, depending on the number of infusion pumps needed, additional docking apparatuses may be added in a stacked configuration. The medical device hub accordingly provides a compact and adaptable infusion management system that requires a relatively small footprint.

The example system, method, and apparatus are configured to detect a position of a medical device within the medical device hub. As disclosed herein, the connectivity apparatus is positioned at a top of the medical device hub. A top of a first docking apparatus is connected to a bottom of the connectivity apparatus. A top of a second docking apparatus may then be connected to a bottom of the first docking apparatus. Additional docking apparatuses are added in a similar manner such that the medical device hub consists of one connectivity apparatus and one or more stacked docking apparatuses. A power cord is connected to the bottom docking apparatus to provide power from a wall outlet or other power source. As such, only a single power cord is needed to power all of the medical devices connected to the medical device hub disclosed herein.

Each docking apparatus includes connectors for receiving two (or more) medical devices. When in use, the medical device hub may include one to six docking apparatuses, with each docking apparatus containing one, two, or more medical devices. The example system, method, and apparatus are configured to determine how many docking apparatuses are connected and how many medical devices are connected to each docking apparatus. Additionally, the example system, method, and apparatus are configured to associate a medical device identifier for each medical device to the position within the docking apparatuses of the medical device hub.

The association between medical devices and their position enables a separate display device, which is communicatively coupled to the medical device hub, to show a graphical representation of each medical device within the medical device hub at the appropriate position. The display device may additionally display medical device data including treatment information, events, alert, or status information in proximity to the graphical representation of the corresponding medical device. A clinician can more easily locate the appropriate medical device within the medical device hub since the position of the medical device corresponds to the position shown within the graphical representation of the medical device hub that is provided by the display device. Locating the position of a medical device quickly may be important to address an alarm or change a fluid container, such as an intravenous (“IV”) bag.

The system, method, and apparatus are configured to determine medical device positions by first determining an order of connected docking apparatuses. To determine an order of connected docking apparatuses, a microcontroller of the connectivity apparatus transmits an enumeration request message along a point-to-point communication connection to a microcontroller of a directly connected docking apparatus. The point-to-point communication connection may be configured for the universal asynchronous receiver/transmitter (“UART”) communication protocol, for example. The enumeration request message includes an initial position value, such as a value of ‘0’.

The connected microcontroller of the docking apparatus receives the enumeration request message and increments the initial position value. For example, the microcontroller may increment the position value to ‘1’. The microcontroller of the docking apparatus then transmits an enumeration response message to the microcontroller of the connectivity apparatus via the point-to-point communication connection. The enumeration response message also includes an identifier of the docking apparatus. The microcontroller of the docking apparatus receives the enumeration response message and associates the position value of ‘1’ with the identifier of the docking apparatus.

When the docking apparatus detects that another docking apparatus is connected, the microcontroller of the docking apparatus transmits a second enumeration request message using a point-to-point communication connection between the docking apparatuses. The second enumeration request message includes the incremented position value. A microcontroller of the other docking apparatus similarly is configured to further increment the position value, such as incrementing the position value to ‘2’. The microcontroller of the other docking apparatus also transmits a second enumeration response message to the docking apparatus. The second enumeration response message includes the further incremented position value. The microcontroller of the docking apparatus is configured to relay the second enumeration response message to the connectivity apparatus, thereby enabling the microcontroller of the connectivity apparatus to record a position and identifier of the other docking apparatus. The process continues until all of the connected docking apparatuses of the medical device hub have reported their positions to the connectivity apparatus.

After the order of the docking apparatuses is determined, the connectivity apparatus is configured to determine positions of medical devices that are connected to each of the docking apparatuses. The microcontroller of the connectivity apparatus transmits trigger messages, or more generally triggers, to each of the microcontrollers of the docking apparatuses using the point-to-point communication connections. The trigger messages cause each microcontroller of the respective docking apparatus to determine a position of each connected medical device using the already determined position value of the docking apparatus. The microcontroller of the docking apparatus then transmits a signal or message to each of the connected medical devices with their determined position. The medical devices are configured to individually transmit a position response message along a communication bus that is also communicatively coupled to the microcontrollers of the docking apparatuses and the connectivity apparatus. The position response message includes the determined position of the medical device in addition to an identifier of the medical device. The microcontroller of the connectivity apparatus receives the response messages and creates a table or other data structure that associates hub positions with the medical device identifiers. At this point, the position of the medical devices is communicated by the connectivity apparatus to monitoring devices or a server to enable the medical devices to be graphically shown in their correct position within the medical device hub.

Aspects of the subject matter described herein may be useful alone or in combination with one or more other aspect described herein. Without limiting the foregoing description, in a first aspect of the present disclosure, a medical device hub includes a connectivity apparatus comprising a memory device storing a data structure of position enumeration data for docking apparatuses and medical devices, a microcontroller communicatively coupled to the memory device of the connectivity apparatus, a first data connector, a first communication bus transceiver communicatively coupled to the microcontroller, a first portion of a communication bus communicatively coupled to the first communication bus transceiver and the first data connector, and a first communication connection provided between the microcontroller and the first data connector. The medical device hub also includes a docking apparatus mechanically coupled to the connectivity apparatus. The docking apparatus comprises a memory device storing an identifier of the docking apparatus, a position value of the docking apparatus, and at least one base slot position, a microcontroller communicatively coupled to the memory device of the docking apparatus, a second data connector connected to the first data connector of the connectivity apparatus, a second communication bus transceiver communicatively coupled to the microcontroller of the docking apparatus, a second portion of the communication bus provided between the second communication bus transceiver and the second data connector, a second communication connection communicatively coupled to the microcontroller of the docking apparatus and the second data connector, a device connector for communicatively coupling to a medical device via a device communication connection that is communicatively coupled to the microcontroller of the docking apparatus, the device connector associated with one of the base slot positions, and a third portion of the communication bus communicatively coupled to the second portion of the communication bus and the device connector. Docking apparatus position enumeration is performed by the microcontroller of the connectivity apparatus transmitting, via the first communication connection, an enumeration request message that includes an initial position value, the microcontroller of the docking apparatus receiving, via the second communication connection, the enumeration request message, incrementing the initial position value, transmitting, via the second communication connection, an enumeration response message that includes the incremented position value and the identifier of the docking apparatus, and storing the incremented position value to the memory device of the docking apparatus, and the microcontroller of the connectivity apparatus receiving, via the first communication connection, the enumeration response message and storing the incremented position value and the identifier of the docking apparatus to the memory device of the connectivity apparatus as position enumeration data. Medical device position enumeration is performed by the microcontroller of the connectivity apparatus transmitting a medical device enumeration trigger, via the first communication connection, responsive to the medical device enumeration trigger, the microcontroller of the docking apparatus determining an overall slot position for a medical device connected to the device connector using the incremented position value and the corresponding base slot position, the microcontroller of the docking apparatus transmitting, via the device communication connection, a signal that is indicative of the overall slot position, causing, the medical device to transmit its medical device identifier and the overall slot position via the communication bus, and the microcontroller of the connectivity apparatus receiving the medical device identifier and the overall slot position via the communication bus and storing the medical device identifier and the overall slot position to the data structure as position enumeration data.

In accordance with a second aspect of the present disclosure, which may be used in combination with any other aspect disclosed herein, the microcontroller of the docking apparatus transmits the signal to the medical device after detecting that the medical device is connected to the device connector of the docking apparatus.

In accordance with a third aspect of the present disclosure, which may be used in combination with any other aspect disclosed herein, the medical device identifier includes at least one of a serial number, a communication bus address, an IP address, or a media access control (“MAC”) address.

In accordance with a fourth aspect of the present disclosure, which may be used in combination with any other aspect disclosed herein, the communication bus includes at least one of a controller area network (“CAN”), single-pair Ethernet, a Fieldbus connection, a Highway Addressable Remote Transducer (“HART”) connection, or a serial connection.

In accordance with a fifth aspect of the present disclosure, which may be used in combination with the first aspect, the docking apparatus position enumeration is additionally performed by: the microcontroller of the docking apparatus detecting another docking apparatus physically connected to the docking apparatus, the microcontroller of the docking apparatus transmitting, via a communication connection with the other docking apparatus, a second enumeration request message that includes the incremented position value, causing a microcontroller of the other docking apparatus to further increment the incremented position value and transmit a second enumeration response message that includes the further incremented position value and the identifier of the other docking apparatus, the microcontroller of the docking apparatus transmitting, via the second communication connection, the second enumeration response message to the connectivity apparatus, and the microcontroller of the connectivity apparatus receiving, via the first communication connection, the second enumeration response message and storing the further incremented position value and the identifier of the other docking apparatus to the memory device of the connectivity apparatus as additional position enumeration data.

In accordance with a sixth aspect of the present disclosure, which may be used in combination with the first aspect, the docking apparatus position enumeration and the medical device position enumeration is performed before the medical device performs a treatment.

In accordance with a seventh aspect of the present disclosure, which may be used in combination with any other aspect disclosed herein, the docking apparatus position enumeration and the medical device position enumeration is performed when the microcontroller of the connectivity apparatus detects the connection of the docking apparatus or detects a connection of a new medical device.

In accordance with an eighth aspect of the present disclosure, which may be used in combination with any other aspect disclosed herein, the docking apparatus position enumeration and the medical device position enumeration is performed when the connectivity apparatus is powered on.

In accordance with a ninth aspect of the present disclosure, which may be used in combination with any other aspect disclosed herein, a medical device hub includes a connectivity apparatus comprising a memory device storing a data structure of position enumeration data for docking apparatuses and medical devices, and a microcontroller communicatively coupled to the memory device of the connectivity apparatus. The medical device hub also includes a docking apparatus mechanically coupled to the connectivity apparatus. The docking apparatus comprises a memory device storing an identifier of the docking apparatus, a position value of the docking apparatus, and at least one base slot position, a microcontroller communicatively coupled to the memory device of the docking apparatus, and a device connector for communicatively coupling to a medical device via a device communication connection that is communicatively coupled to the microcontroller of the docking apparatus, the device connector associated with one of the base slot positions. Docking apparatus position enumeration is performed by the microcontroller of the connectivity apparatus transmitting an enumeration request message that includes an initial position value, the microcontroller of the docking apparatus receiving the enumeration request message, incrementing the initial position value, transmitting an enumeration response message that includes the incremented position value and the identifier of the docking apparatus, and storing the incremented position value to the memory device of the docking apparatus, and the microcontroller of the connectivity apparatus receiving the enumeration response message and storing the incremented position value and the identifier of the docking apparatus to the memory device of the connectivity apparatus as position enumeration data. Medical device position enumeration is performed by the microcontroller of the connectivity apparatus transmitting a medical device enumeration trigger, responsive to the medical device enumeration trigger, the microcontroller of the docking apparatus determining an overall slot position for a medical device connected to the device connector using the incremented position value and the corresponding base slot position, the microcontroller of the docking apparatus transmitting a signal that is indicative of the overall slot position, causing, the medical device to transmit its medical device identifier and the overall slot position, and the microcontroller of the connectivity apparatus receiving the medical device identifier and the overall slot position and storing the medical device identifier and the overall slot position to the data structure as position enumeration data.

In accordance with a tenth aspect of the present disclosure, any of the structure and functionality illustrated and described in connection with FIGS. 1 to 15 may be used in combination with any of the structure and functionality illustrated and described in connection with any of the other of FIGS. 1 to 15 and with any one or more of the preceding aspects.

In light of the present disclosure and the above aspects, it is therefore an advantage of the present disclosure to provide a medical device hub that determines positions of connected medical devices.

It is another advantage of the present disclosure to automatically determine a number of docking apparatus and medical devices connected to a hub.

It is a further advantage of the present disclosure to use positions of medical devices within a hub to accurately, graphically depict positions of the medical devices within one or more user interfaces.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a perspective view of a medical device hub with a single docking apparatus and a connectivity apparatus, according to an example embodiment of the present disclosure.

FIG. 2 is a diagram of an assembly view of the medical device hub of FIG. 1, according to an example embodiment of the present disclosure.

FIG. 3 shows a bottom view of the medical device hub of FIG. 1, according to an example embodiment of the present disclosure.

FIG. 4 is a diagram of a medical device hub with six docking apparatuses, according to an example embodiment of the present disclosure.

FIG. 5 is a diagram of the medical device hub of FIGS. 1 to 3 with two medical devices connected to the docking apparatus, according to an example embodiment of the present disclosure.

FIG. 6 is a diagram of the medical device hub communicatively coupled to a gateway server and a monitoring device, according to an example embodiment of the present disclosure.

FIG. 7 illustrates an example graphical representation of the medical device hub of FIGS. 1 to 6, according to an example embodiment of the present disclosure.

FIG. 8 is a diagram that is illustrative of power routing within the docking apparatus of the medical device hub, according to an example embodiment of the present disclosure.

FIG. 9 is a diagram that shows communication connections within the docking apparatus of FIGS. 1 to 6, according to an example embodiment of the present disclosure.

FIG. 10 is a diagram of a device detection circuit located on a printed circuit board of the docking apparatus, according to an example embodiment of the present disclosure.

FIG. 11 is a diagram of an apparatus detection circuit of the docking apparatus, according to an example embodiment of the present disclosure.

FIG. 12 is a diagram of communication connections provided by the connectivity apparatus, according to an example embodiment of the present disclosure.

FIG. 13 shows a flow diagram illustrating an example procedure for detecting positions of medical devices connected to the medical device hub, according to an example embodiment of the present disclosure.

FIG. 14 is a diagram illustrative of performing docking apparatus position enumeration, according to an example embodiment of the present disclosure.

FIG. 15 shows a diagram of medical device position enumeration performed by the connectivity apparatus in conjunction with the docking apparatuses, according to an example embodiment of the present disclosure.

### DETAILED DESCRIPTION

The present disclosure relates in general to a method, system, and apparatus for determining positions of medical devices within a hub. As disclosed herein, the medical device hub comprises a number of stages that are linked together in a stacked configuration. Each medical device hub includes a single connectivity apparatus stage (more generally referred to as a connectivity apparatus) that is communicatively coupled to a medical network and/or a monitoring device. Additionally, each medical device hub includes one or more docking apparatus stages (more generally referred to as docking apparatuses). Each docking apparatus can accommodate, in some embodiments, two or more medical devices, such as infusion pumps. The number of docking apparatuses used in the medical device hub depends on the number of medical devices needed for a patient treatment. Together, the connectivity apparatus and the docking apparatuses enable medical device data from the medical devices to be routed to a medical network and/or a monitoring device.

In contrast to the medical device hub disclosed herein, known multi-channel infusion pumps include a single controller and two to six pumps. The controller provides centralized management of the infusion pumps such that the pumps cannot be removed during operation. The controller uses an internal network to manage pump position. Additionally, the controller is configured to operate with only one type of infusion pump, thereby limiting system flexibility.

The medical device hub overcomes at least some of the limitations of known multi-channel infusion pumps by enabling multiple docking apparatuses to be stacked as needed, where each docking apparatus can accommodate one of many different types of medical devices. Additionally, the medical devices are configured to operate independently and may be removed from the medical device hub as needed, even during a treatment without interruption. Such a configuration provides a scalable, flexible, and adaptable system that aggregates medical devices into a relatively small footprint.

### Medical Device Hub Embodiment

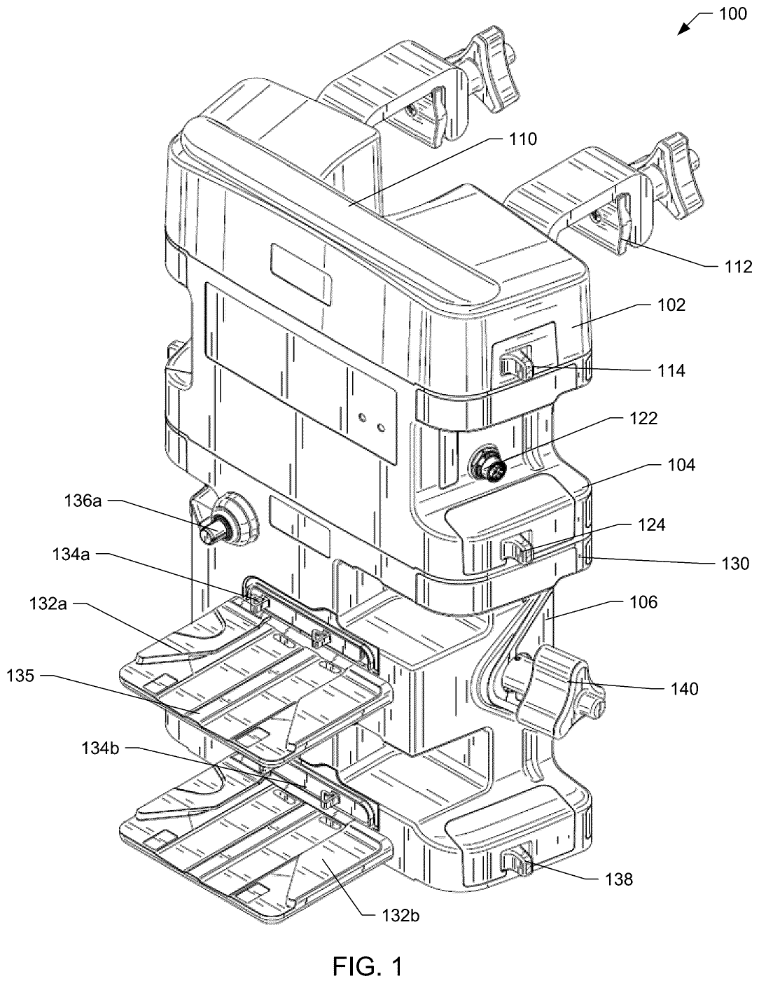

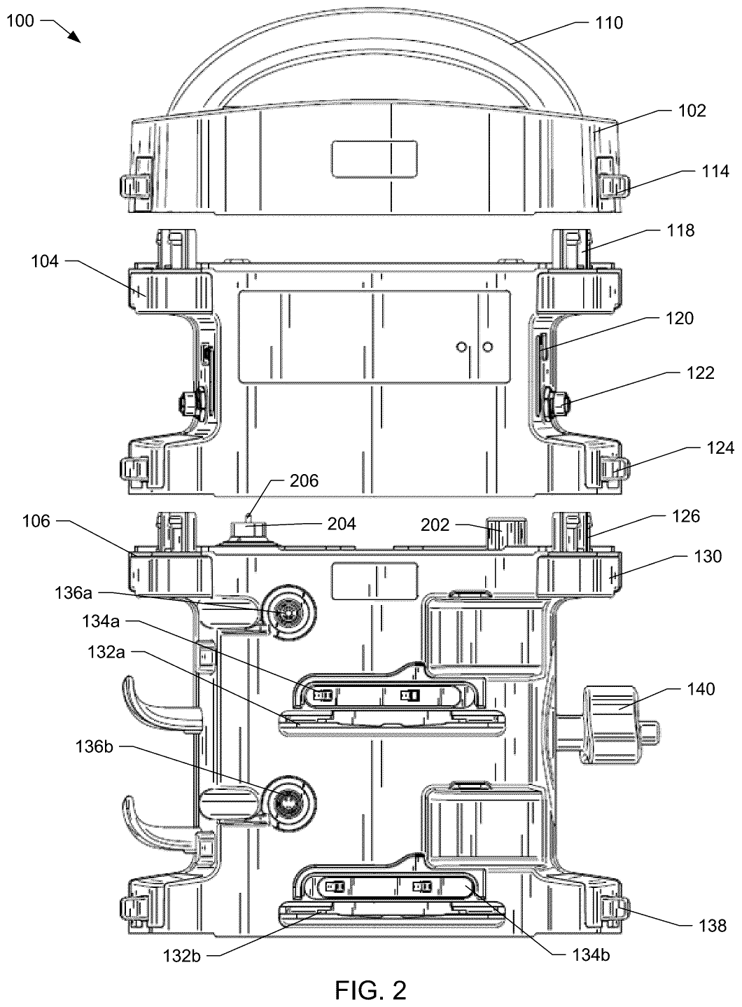

FIGS. 1 and 2 are diagrams of a medical device hub 100, according to an example embodiment of the present disclosure. FIG. 1 shows the medical device hub 100 from a perspective view. FIG. 2 shows the medical device hub 100 from a front assembly view.

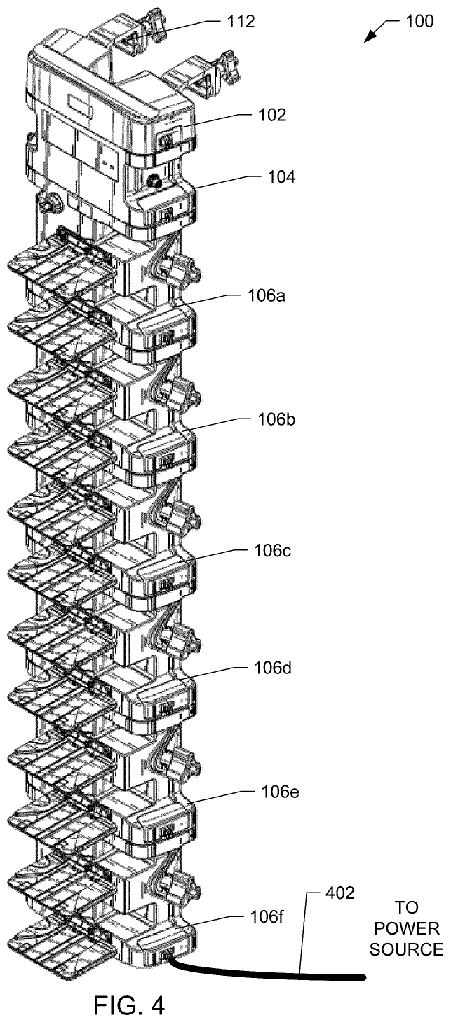

The medical device hub 100 includes a handle stage 102, a connectivity apparatus 104, and a docking apparatus 106. While only one docking apparatus 106 is shown in FIGS. 1 and 2, the medical device hub 100 can include additional docking apparatuses 106, as shown in FIG. 4. In some embodiments, the handle stage 102 may be omitted.

As shown in FIGS. 1 and 2, the handle stage 102 includes a handle 110, a bedrail clamp 112, and a release lever 114. The handle 110 is configured to enable the medical device hub 100 to be carried by a clinician. The example handle 110 has a semi-circular shape that extends vertically from a housing of the handle stage 102. The handle 110 may be connected to or integrally formed with the housing of the handle stage 102 to support the weight of the connectivity apparatus 104 and the one or more docking apparatuses 106 during transportation to other locations within a hospital or other medical environment.

The bedrail clamp 112 includes a bracket and a release knob to enable the medical device hub 100 to be connected to a railing or a panel of a patient's bed. The release knob is rotated in one direction to cause a screw or other actuator to move to a closed position, thereby tightening against a rail or hospital bed panel. The release knob is rotated in an opposite direction to cause the screw or other actuator to move to a closed position. In some embodiments, the bedrail clamp 112 may be omitted.

The release lever 114 is configured, when actuated by a clinician, to enable the handle stage 102 to be separated from the connectivity apparatus 104. As shown in FIG. 2, the connectivity apparatus 104 includes a protrusion section 118 that is configured to mate with a recess section within the handle stage 102. The protrusion section 118 may include a tab or other mechanical connector that engages the corresponding release lever 114 and slots or protrusions within the recess section of the handle stage 102. Insertion of the protrusion section 118 into the recess section may create a secure connection between the handle stage 102 and the connectivity apparatus 104 when the release lever 114 is engaged. However, when the release lever 114 is actuated, the protrusion section 118 may be slid from the recess section of the handle stage 102, thereby enabling separation of the connectivity apparatus 104. While FIGS. 1 and 2 show the release lever 114 located on both sides of the handle stage 102, in other embodiments the release lever 114 is located only on one side of the handle stage 102.

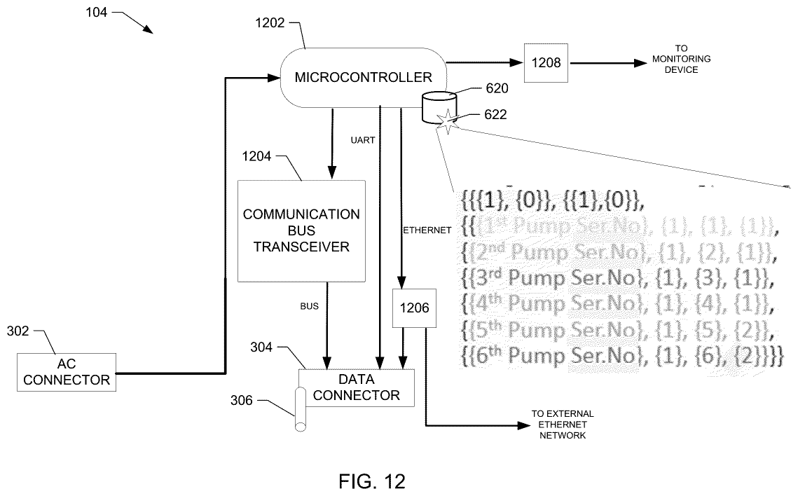

The connectivity apparatus 104 is configured to provide communication between the medical device hub 100 and a hospital network or a monitoring device. The communication configuration of the connectivity apparatus 104 is discussed in more detail in conjunction with FIG. 12. A housing of the connectivity apparatus 104 includes an Ethernet port 120 (shown in FIG. 2), such as an RJ45 port. The connectivity apparatus 104 also includes one or more M12 connectors 122. In some embodiments, the M12 connector 122 is omitted. Similar to the handle stage 102, the connectivity apparatus 104 also includes a release lever 124 to enable the docking apparatus 106 to be removably connected via similar protrusion sections 126.

The docking apparatus 106 is configured to be connected to a bottom of the housing of the connectivity apparatus 104, as shown in FIGS. 1 and 2. In the illustrated example, the docking apparatus 106 is configured to connect to two medical devices, such as infusion pumps. In other embodiments, the docking apparatus 106 may include a single shelf and connector to couple to only one medical device or have as many as six shelves and six connectors to couple to six medical devices.

The docking apparatus 106 includes a housing 130 and shelves 132a, 132b that extend from the housing 130. Latches 134a, 134b are respectively positioned above each of the shelves 132a, 132b. The latches 134a, 134b are configured to releasably couple to respective medical devices, such as infusion pumps. The shelves 132a, 132b are configured to support a weight of a respective medical device to ensure the medical device does not break away from the respective latch 134a, 134b. The width of the shelves 132a, 132b is configured to correspond to a width of a largest medical device that is to be connected to the docking apparatus 106 such that the largest medical device does not have a significant portion that extends beyond the width of the shelves 132a, 132b. The shelves have a length that is between 50% to 75% of a length of the docking apparatus 106 to provide sufficient support for a connected medical device. Additionally, as shown in FIGS. 1 and 2, the shelves 132a, 132b include recessed sections 135 to guide the placement of a medical device into the respective latch 134a, 134b.

The docking apparatus 106 also includes two device connectors 136a, 136b that extend from the housing 130. The device connectors 136a, 136b are located on the housing 130 of the docking apparatus 106 so as to mate with respective connectors that are located on compatible medical devices. In the illustrated example, the device connectors 136a, 136b have a cylindrical shape that protrudes from the housing 130. In other examples, the device connectors 136a, 136b may be ports that are recessed into the housing 130 and/or have a rectangular or hexagonal shape. Further, while the device connectors 136a, 136b are shown as being located above the respective latches 134a, 134b, in other embodiments, they may be offset from the latches 134a, 134b.

The device connectors 136a, 136b are configured to include pins or other contacts for power and data routing between a medical device and the docking apparatus 106. In an example, the device connectors 136a, 136b include a device-present pin for detecting the connection of a medical device. Circuitry within the medical device may apply a low voltage or ground to the device-present pin when the medical device is connected, for example, to the device connector 136a. The docking apparatus 106 is configured to detect when the device-present pin receives a low-voltage or ground to determine when the medical device is connected.

The device connectors 136a, 136b may also include pins for a controller area network (“CAN”) connection (e.g., a CAN high signal pin and a CAN low signal pin) and/or an Ethernet connection (e.g., positive and negative transmission and receiving signal pins). In some embodiments, the device connectors 136a, 136b may include an identifier request pin. In these embodiments, the identifier request pin is used by the docking apparatus 106 and/or the connectivity apparatus 104 to transmit a signal or a message requesting that the medical device provide a CAN identifier (e.g., a CAN identification Query ID) or a medical device identifier through the CAN connection.

While the disclosure references CAN connections, it should be appreciated that other connections can be used. For example, a single-pair Ethernet connection, a Fieldbus connection, a Highway Addressable Remote Transducer (“HART”) connection, or a serial connection may be used. Alternatively, a wireless connection, such as Bluetooth® or Zigbee™ may be used.

FIGS. 1 and 2 also show that the housing 130 of the docking apparatus 106 includes a release lever 138, which is similar to the release lever 124 of the connectivity apparatus 104. Similar to the respective connections between the handle stage 102 and the connectivity apparatus 104 and the connectivity apparatus 104 and the docking apparatus 106, the docking apparatuses 106 may be removably connected to each other in a stacked arrangement. The release lever 138 enables the docking apparatus 106 to be disconnected from a lower-positioned docking apparatus.

As discussed above, the medical device hub 100 is configured to route power and data through the stack of the connectivity apparatus 104 and one or more docking apparatuses 106. FIG. 2 shows connectors that are positioned at a top of the housing 130 of the docking apparatus 106. The connectors include an AC outlet connector 202 and a top data connector 204. The AC outlet connector 202 is configured to protrude from a top of the housing 130 and may include an International Electrotechnical Commission (“IEC”) connector with countersunk holes. The outlet connector 202 may have 10 ampere, 250 AC voltage rating.

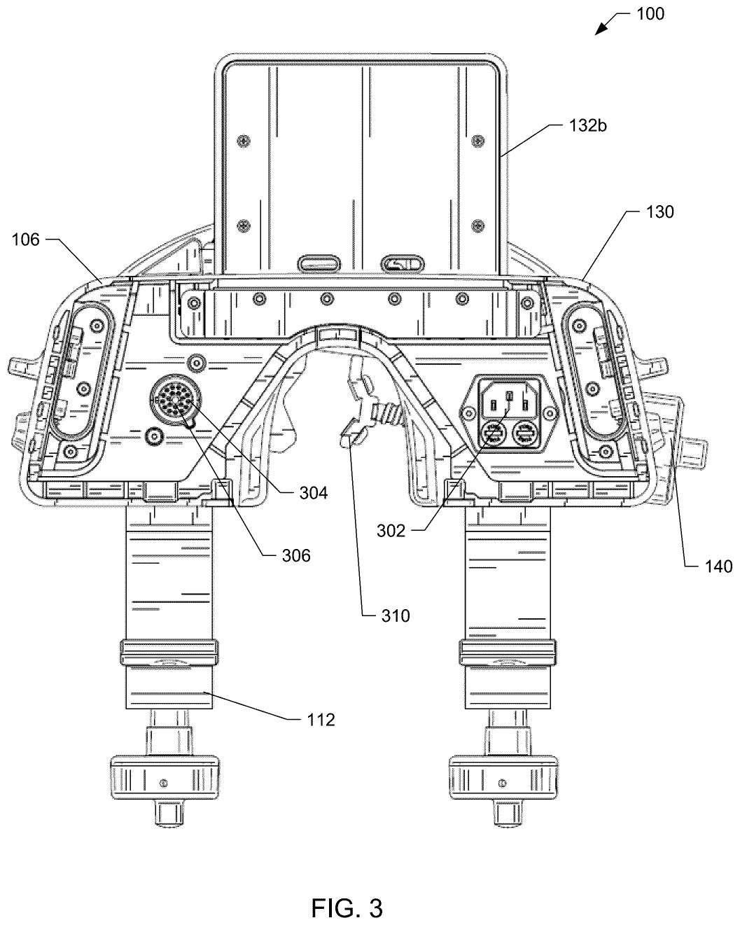

The AC outlet connector 202 is configured to connect to a corresponding AC inlet connector that is provided at a bottom of the connectivity apparatus 104 and other docking apparatuses 106. FIG. 3 shows a bottom view of the docking apparatus 106, according to an example embodiment of the present disclosure. It should be appreciated that the bottom view is similar for the connectivity apparatus 104. As shown, a bottom of the housing 130 of the docking apparatus 106 includes an AC inlet connector 302 that is aligned with the AC outlet connector 202 at the top of the housing. The AC inlet connector 302 may include a panel mounted appliance inlet that sits within a recess section of the docking apparatus 106. When connected, the prongs of the AC inlet connector 302 slide into the apertures of the AC outlet connector 202 as a body of the AC outlet connector 202 fits within a recess of the AC inlet connector 302.

The aligned positioning of the AC inlet connector 302 and the AC outlet connector 202 enable the connectivity apparatus 104 and the docking apparatuses 106 to be electrically connected together when stacked within the medical device hub 100. Further, the consistent positioning of the AC inlet connector 302 and the AC outlet connector 202 enable any docking apparatus 106 to be connected to any other docking apparatus 106 or the connectivity apparatus 104. In some alternative embodiments, the AC inlet connector 302 may be positioned on the top of the housing 130 while the AC outlet connector 202 is positioned on the bottom of the housing 130 of the docking apparatus 106. Further, while the connectors 202 and 302 are shown as Type-B connectors for use in North America, in other examples, the connectors may be of Type C through Type 0 to enable the medical device hub 100 to be used in other parts of the world.

To power the medical device hub 100, a power cord is connected to the AC inlet connector 302 at the bottom of a lower-most docking apparatus 106. An opposite end of the power cord may be connected to an electrical outlet, a power rail, a battery, a generator, or any other power source. Such a configuration ensures that the power cord is placed as close to the ground as possible to reduce the number of wires and lines at higher sections of the hub 100. Further, the routing of power through the medical device hub 100 means that only a single power cord is needed.

In addition to routing power throughout, the medical device hub 100 is also configured to enable data to be communicated among the connectivity apparatus 104 and the docking apparatuses 106. FIG. 2 shows the top data connector 204 adjacent to the AC outlet connector 202 at the top of the housing 130 of the docking apparatus 106. FIG. 3 shows a bottom data connector 304 that is located at the bottom of the housing 130 adjacent to the AC inlet connector 302. The top data connector 204 is configured to connect to a bottom data connector 304 of the connectivity apparatus 104 or the bottom data connector 304 of another docking apparatus 106.

The data connectors 204 and 304 may include pins for CAN communication, for example. In some embodiments, the data connectors 204 and 304 may additionally include pins for universal asynchronous receiver-transmitter (“UART”) communication and/or Ethernet communication between the connectivity apparatus 104 and the stacked docking apparatuses 106 within the medical device hub 100. The data connectors 204 and 304 each includes at least one respective pin 206, 306 to detect when the connectivity apparatus 104 or another docking apparatus 106 is connected. In some embodiments, the pin 306 of the bottom data connector 304 is electrically coupled to ground or a low voltage. As described in more detail below, a circuit with the docking apparatus 106 is configured to detect when the pin 206 of the top data connector 204 is pulled to the ground or low voltage to detect when another docking apparatus 106 or the connectivity apparatus 104 is connected.

FIGS. 1 to 3 also show that the docking apparatus 106 includes a pole clamp, which includes a knob 140 and an actuator 310. The pole clamp enables the docking apparatus 106 to securely couple the medical device hub 100 to a pole. Since each docking apparatus 106 includes a pole clamp, multiple stacked docking apparatuses 106 are connected to the same pole to securely connect the medical device hub 100 when a plurality of medical devices are used. To connect, the knob 140 is rotated, which causes the actuator 310 to engage the pole. To release, the knob 140 is rotated in an opposite direction, which causes the actuator 310 to move away from the pole.

FIG. 4 shows a diagram of the medical device hub 100 of FIG. 1 with six docking apparatuses 106a to 106f stacked below the connectivity apparatus 104, according to an example embodiment of the present disclosure. In the illustrated example, the handle stage 102 is provided at the top of the medical device hub 100. The connectivity apparatus 104 is connected to the bottom of the handle stage 102. A first docking apparatus 106a is connected to a bottom of the connectivity apparatus 104. A second docking apparatus 106b is connected to a bottom of the first docking apparatus 106a. Further, third through sixth docking apparatuses 106c to 106f are sequentially stacked. A power cord 402 electrically couples the sixth docking apparatus 106f to a power source, such as a wall outlet.

The illustrated six docking apparatuses 106a to 106f are configured to accommodate twelve medical devices. To support the weight of the docking apparatuses 106a to 106f themselves in addition to the medical devices, each of the docking apparatuses 106a to 106f includes pole clamps for connecting to a pole. In the alternative, the bedrail clamp 112 may connect to a bedrail to support the medical device hub 100.

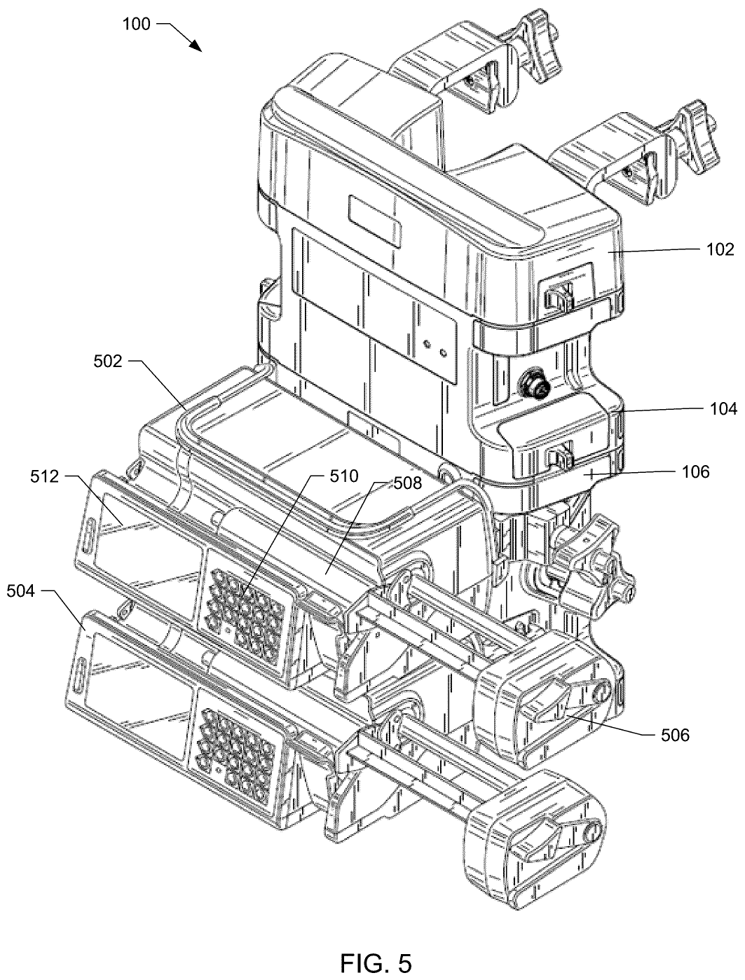

FIG. 5 is a diagram of the medical device hub 100 of FIGS. 1 to 3 with two medical devices 502, 504 connected to the docking apparatus 106, according to an example embodiment of the present disclosure. As shown, a first medical device 502 is slide across the shelf 132a to engage the latch 134a. As the medical device 502 is being connected, the device connector 136a of the docking apparatus 106 contacts a corresponding connector of the medical device 502. In a similar manner, a second medical device 504 is slid across the shelf 132b to engage the latch 134b. As the medical device 504 is being connected, the device connector 136b of the docking apparatus 106 contacts a corresponding connector of the medical device 504.

In the illustrated example, the medical devices 502 and 504 are syringe pumps, which include an actuator 506 that presses on a plunger of a syringe 508 to dispense a fluid into an IV tube. The actuator 506 is controlled by a motor within a housing of the medical device 502. Additionally, the medical device 502 includes a user interface comprising a keypad 510 and a display screen 512. The keypad 510 includes one or more buttons or switches for controlling operation of the medical device 502. The display screen 512 displays graphics and text regarding the operation of the medical device 502. In some embodiments, the display screen 512 may be a touchscreen. In some instances, a clinician uses the keypad 510 and the display screen 512 to program an infusion therapy. In addition to manual programming, the medical device 502 may receive electronic prescriptions from a hospital information system via a network. The medical device 502 may include one or more drug libraries that include particular limits based on a care area, a dose, a rate of change, a drug type, a drug concentration, a patient age, a patient weight, etc.

As an infusion pump, the medical device 502 is configured to perform an infusion therapy for a patient, which includes the infusion of one or more fluids, solutions, or drugs into the patient. The medical device 502 operates according to an infusion prescription entered by a clinician at the keypad 510 and/or display screen 512 or received via a network. The medical device 502 may compare the prescription to the drug library and provide any alerts or alarms when a parameter of the prescription violates a soft or hard limit. The medical device 502 is configured to monitor the progress of the therapy and periodically transmit medical device data to a gateway server. The medical device data may include, for example, an infusion rate, a dose, a total volume infused, a time remaining for the therapy, a fluid concentration, a rate change, a volume remaining within a medication container, a fluid name, a patient identifier, titration information, bolus information, a care area identifier, a timestamp when the data was generated, an alarm condition, an alert condition, an event, etc. In some instances, the medical device data includes a new infusion start event including information indicative of an infusion pump identifier, an infused fluid name, an infusion rate, a volume to be infused, a dose, a volume remaining, and/or a time the new infusion start event was generated by the infusion pump. The medical device 502 may transmit the data continuously, periodically (e.g., every 30 seconds, 1 minute, etc.), or upon request by a gateway server. While a syringe pump is shown, the docking apparatus 106 may also connect to other types of infusion pumps such as linear peristaltic pumps, large volume parenteral pumps, ambulatory pumps, PCA pumps, multi-channel pumps, etc.

The medical devices 502 and 504 may also include renal failure therapy (“RFT”) machines, which may include any hemodialysis, hemofiltration, hemodiafiltration, continuous renal replacement therapy (“CRRT”), or peritoneal dialysis machine. CRRT is a dialysis modality typically used to treat critically ill, hospitalized patients in an intensive care unit who develop acute kidney injury (“AKI”). Unlike chronic kidney disease, which occurs slowly over time, AKI often occurs in hospitalized patients and typically occurs over a few hours to a few days. A patient, undergoing hemodialysis, for example, is connected to the RFT machine, where the patient's blood is pumped through the machine. The blood passes through a dialyzer of the machine, which removes waste, toxins and excess water (e.g., ultrafiltrate) from the blood. The cleaned blood is returned to the patient.

Hemodialysis is a renal failure treatment in which waste from the blood is diffused across a semi-permeable membrane. During hemodialysis, blood is removed from the patient and flows through a semi-permeable membrane assembly (dialyzer), where the blood flows generally counter-current to dialysis solution flowing on the other side of the semipermeable membrane. In the dialyzer, toxins from the blood travel across the semi-permeable membrane and exit the dialyzer into used dialysis solution (dialysate). The cleaned blood having flowed through the dialyzer is then returned to the patient.

Hemofiltration is another renal failure treatment, similar to hemodialysis. During hemofiltration, a patient's blood is also passed through a semipermeable membrane (a hemofilter), where fluid (including waste products) is pulled across the semipermeable membrane by a pressure differential. This convective flow brings certain sizes of molecular toxins and electrolytes (which are difficult for hemodialysis to clean) across the semipermeable membrane. During hemofiltration, a replacement fluid is added to the blood to replace fluid volume and electrolytes removed from the blood through the hemofilter. Hemofiltration in which replacement fluid is added to the blood prior to the hemofilter is known as pre-dilution hemofiltration. Hemofiltration in which replacement fluid is added to the blood after the hemofilter is known as post-dilution hemofiltration.

The RFT machine can alternatively be a hemodiafiltration machine. Hemodiafiltration is a further renal failure treatment that uses hemodialysis in combination with hemofiltration. Blood is again pumped through a dialyzer, which accepts fresh dialysis fluid unlike a hemofilter. With hemodiafiltration, however, replacement fluid is delivered to the blood circuit, like with hemofiltration. Hemodiafiltration is accordingly a neighbor of hemodialysis and hemofiltration.

Alternatively, the medical device 502 may be a peritoneal dialysis machine, which may perform various types of peritoneal dialysis therapies, including continuous cycling peritoneal dialysis (“CCPD”), tidal flow automated peritoneal dialysis (“APD”), and continuous flow peritoneal dialysis (“CFPD”). Peritoneal dialysis infuses dialysate into a patient during fill cycles.

For any dialysis treatment, the RFT machine may compare parameters of a prescription to one or more limits and provide any alerts or alarms when a parameter of the prescription violates a soft or hard limit. The RFT machine is configured to monitor the progress of the therapy and periodically transmit medical device data to a gateway server. The medical device data may include, for example, a fill rate, a dwell time, a drain or fluid removal rate, a blood flow rate, an effluent dose, an ultrafiltration removal rate, a dialysate removal rate, a total dialysate infused, a dialysate flow, a replacement pre-flow, a replacement post-flow, a patient weight balance, a return pressure, an excess patient fluid sign, a filtration fraction, a time remaining, a dialysate concentration, a dialysate name, a patient identifier, a room identifier, a care area identifier, a timestamp when the data was generated, an alarm condition, an alert condition, an event, etc. The RFT machine may transmit the data continuously, periodically (e.g., every 30 seconds, 1 minutes, etc.), or upon request by the gateway server.

In some embodiments, the medical device 502 may include a physiological sensor. For example, the medical device 502 may include a pulse oximetry sensor. Additionally or alternatively, the physiological sensor may include a blood pressure sensor, a patient weight scale, a glucose sensor, a cardiac monitor, etc. In some instances, instead of connecting directly to the docking apparatus 106, the physiological sensor connects to the medical device 502 via a wired or wireless connection. Physiological data from the sensor is provided as medical device data.

In further embodiments, the docking apparatus 106 may also connect to a hemodynamic monitor, which is configured to display information relevant to hemodynamic monitoring and management. This includes fluid balance information, hemodynamic assessment information, hemodynamic parameters, and/or alerts related to infiltration, infusion line occlusions, and/or a fluid bag being near-empty or empty. The hemodynamic monitor uses therapy progress data and/or dialysis therapy progress data in addition to physiological sensor data (collectively referred to as medical device data) to determine and/or display hemodynamic information.

### Hub Connectivity Station Communication Routing Embodiment

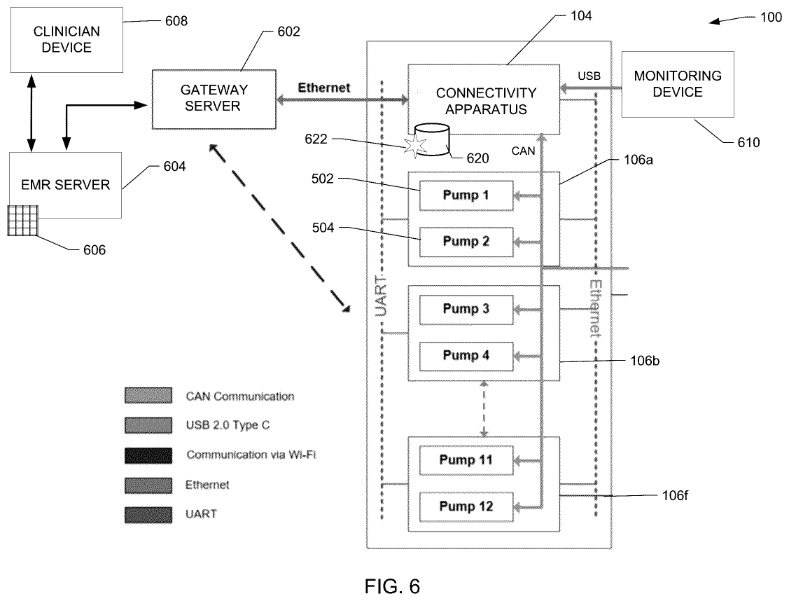

The connectivity apparatus 104 of the example medical device hub 100 of FIGS. 1 to 5 is configured to connect to a network and/or a monitoring device. FIG. 6 is a diagram of the medical device hub 100 communicatively coupled to a gateway server 602, according to an example embodiment of the present disclosure. The gateway server 602 includes a controller, processor, router, switch, computer, etc. configured to communicate with the medical device hub 100 via a network (e.g., a wide area network, a local area network, a wireless local area network, an Ethernet, the Internet, a cellular network, or combinations thereof). The gateway server 602 may be communicatively coupled to more than one medical device hub 100. Further, the gateway server 602 may communicate with other medical devices, such as infusion pumps and RFT machines. The gateway server 602 is configured to provide bi-directional communication with the medical device hub 100 for the wired/wireless secure transfer of drug libraries and medical device data. The gateway server 602 may also be configured to integrate with a hospital information system to transmit the medical device data from the medical devices 502, 504 to a hospital electronic medical record (“EMR”) that is managed by an EMR server 604.

FIG. 6 shows that when, for example, the Ethernet connection between the connectivity apparatus 104 and the gateway server 602 is not available, each of the medical devices 502, 504 may individually communicate with the gateway server 602 via a wireless connection, such as Wi-Fi. The medical devices 502, 504 are configured to detect when bus connections, such as CAN connections, to the connectivity apparatus 104 are not available. Further, the medical devices 502, 504 may detect that the Ethernet connection is not available when messages cannot be delivered to the gateway server 602. When the bus connections and/or the Ethernet connections are not available, medical devices 502, 504 have a fail-safe mode that activates Wi-Fi transponders for communicating wirelessly with a hospital network, for example, to connect to the gateway server 602.

Further, when the medical devices 502, 504 are disconnected from a docking apparatus 106, they are configured to communicate with the gateway server 602 via the Wi-Fi connection. Further, when the medial device hub 100 is powered off (but still connected to a power source), the connectivity apparatus 104 is deactivated, causing the medical devices 502, 504 to communicate directly with the gateway server 602 via, for example, the Wi-Fi connection. The connectivity apparatus 104 may include a switch that enables the medical device hub 100 to be powered off.

The example EMR server 604 is configured to manage patients' EMRs, which are stored in an EMR database 606. The EMR server 604 receives the medical device data and uses a device identifier and/or patient identifier associated with the data to determine a corresponding patient EMR within the EMR database 606. The EMR server 604 is configured to write the medical device data to the appropriate patient EMR within the database 606, thereby providing a record that is accessible to clinicians and medical devices.

In some embodiments, a clinician device 608 is communicatively coupled to the EMR server 604. The clinician device 608 may include a smartphone, a tablet computer, a laptop computer, a desktop computer, a workstation, or a server. The clinician device 608 may be communicatively coupled to the EMR server 604 via a network, for example. The clinician device 608 includes one or more user interfaces for at least viewing the medical device data stored in patient EMRs of the database 606.

In addition to connecting to the gateway server 602, the connectivity apparatus 104 may also include a connection interface for connecting directly to a monitoring device 610. In the illustrated example, the connection interface includes a universal serial bus (“USB”) interface for a wired connection to the monitoring device 610. In other examples, the connection interface may include a micro-USB connection, a serial connection, an HDMI connection, or an Ethernet connection. The direct connection enables the monitoring device 610 to receive medical device data from the medical devices 502, 504 without needing the gateway server 602 and/or the EMR server 604. The monitoring device 610 may include a patient bedside monitor, a hemodynamic monitor, or any other display device configured to present medical device data.

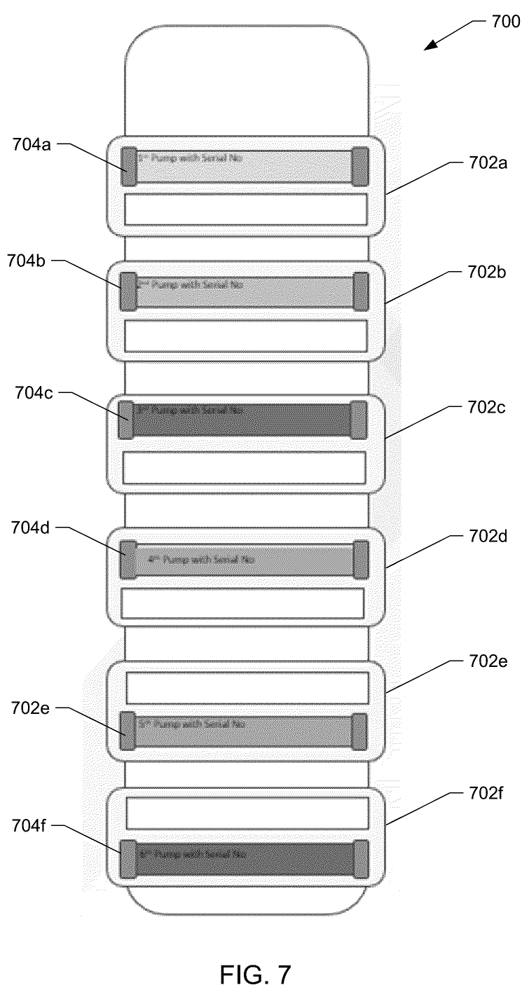

The clinician device 608, the gateway server 604, and/or the monitoring device 610 are configured to display a graphical representation of the medical device hub 100. FIG. 7 illustrates an example graphical representation 700 of the medical device hub 100, according to an example embodiment of the present disclosure. The graphical representation 700 may be displayed in one or more user interfaces displayed by the clinician device 608, the gateway server 604, and/or the monitoring device 610.

The graphical representation 700 shown in FIG. 7 includes graphical representations of six docking apparatus 702a to 702f. The graphical representation 700 also shows locations of medical devices within each of the six docking apparatus 702a to 702f. For example, a graphical representation of a medical device 704a is shown as being located in a top slot of the graphical representation of the docking apparatus 702a. Further, graphical representation 704b, 704c, and 704d are also shown as being located in top slots of the graphical representations of the respective docking apparatuses 702b, 702c, and 702d. A graphical representation of medical devices 704e and 704f are shown as being located in a bottom slot of the graphical representation of the docking apparatuses 702e and 702f.

In the illustrated example, the graphical representations of the medical devices 704a to 704f each includes a serial number or other identifier of the respective medical device. In some examples, the graphical representations of the medical devices 704a to 704f may also include at least some of the medical device data described above. For example, the graphical representations of the medical devices 704a to 704f may display a current status, any alarms/alerts generated, a volume remaining to be infused, a volume until a container is empty, a name of a fluid being infused, and/or an infusion rate, for example. Further, at least some of the graphical representations of the medical devices 704a to 704f may change color based on status or may be colored differently for differentiation.

As discussed herein, the connectivity apparatus 104 includes a memory device 620, as shown in FIG. 6. The memory device 620 is configured to store a data structure of position enumeration data 622 for docking apparatuses and medical devices. The position enumeration data 622 specifies, for example, a top-down order of the docking apparatus 106 in conjunction with identifiers of the docking apparatuses 106, in some embodiments. The position enumeration data 622 also specifies positions of the medical devices among the docking apparatuses 106 in addition to an identifier for each of the medical devices. The identifier may include a serial number, a CAN identifier, and/or a MAC address. The positions of the medical devices may be indicated by an overall slot value within the medical device hub 100.

The connectivity apparatus 104 transmits the position enumeration data 622 to the clinician device 608, the gateway server 604, and/or the monitoring device 610 to enable display of the graphical representation 700. The position enumeration data 622 may be populated into fields for a template of the graphical representation 700, which causes the graphical representation 700 to show a current configuration of the medical device hub 100. For example, when there are only two docking apparatuses 702 populated with position enumeration data 622, the graphical representation 700 only shows the graphical representations of the docking apparatuses 702a and 702b while omitting the graphical representations of the docking apparatuses 702c to 702f. Further, graphical representations of the medical devices 704 are only shown when position enumeration data 622 for that medical device is provided. Otherwise, the graphical representation 700 shows the slot as being empty.

The connectivity apparatus 104 may transmit the position enumeration data 622 directly to the monitoring device 610, for example, in conjunction with the medical device data. For networked devices, the connectivity apparatus is configured to transmit the position enumeration data 622 to the EMR server 604 for storage in a patient's EMR stored in the database 606. Alternatively, the connectivity apparatus 104 receives a request from the clinician device 608 indicative that user interface with the graphical representation 700 is being displayed. In response, the connectivity apparatus 104 transmits the position enumeration data 622 to enable the graphical representation 700 to be properly configured. The clinician device 608 may obtain a network address of the connectivity apparatus 104 from the patient's EMR. The network address of the connectivity apparatus 104 may be stored to the patient's EMR when the medical device data from the medical device hub 100 is transmitted to the patient's EMR. In alternative embodiments, the connectivity apparatus 104 stores the graphical representation 700, which may be transmitted in conjunction with medical device data. In these alternative embodiments, the connectivity apparatus 104 may configure the graphical representation 700 based on the position enumeration data 622.

In some embodiments, the medical devices may include an LED circuit that enables different colors to be emitted. In these embodiments, the colors may be assigned by the connectivity apparatus 104 based on a detected location within the medical device hub 100. The connectivity apparatus 104 may also store the assigned color with the position enumeration data 622. The color may be provided with the position enumeration data 622 for the graphical representation 700 to the clinician device 608, the gateway server 604, and/or the monitoring device 610 such that the color emitted by the medical device corresponds to the color shown in the graphical representation 700.

In some embodiments, a clinician may desire to verify the graphical representation of the medical devices 704 is correct or may desire to quickly locate a medical within the medical device hub 100. A clinician may use a touchscreen or pointer of the clinician device 608, the gateway server 604, and/or the monitoring device 610, which causes a message to be transmitted to the connectivity apparatus 104. The example connectivity apparatus 104 is configured, in these embodiments, to determine which medical device was selected and accordingly transmit a CAN and/or serial communication message to the corresponding medical device. The message may indicate that the corresponding medical device illuminate an LED, blink an LED, flash a display screen (e.g., the display screen 512), and/or emit an audible sound.

As shown in FIG. 6, the connectivity apparatus 104 is communicatively coupled to the docking apparatuses 106a to 106f and corresponding medical devices, such as medical devices 502, 504 via a CAN (bus) connection and/or a serial connection, such as a universal asynchronous receiver/transmitter (“UART”) connection. The connectivity apparatus 104 may also be communicatively coupled to medical devices within the medical device hub 100 via an Ethernet connection.

As discussed in further detail below, the connectivity apparatus 104 determines medical device positions within each docking apparatus 106 and an order of the stacked docking apparatuses 106a to 106f using messaging. The connectivity apparatus 104 then associates identifiers of the medical devices and/or docking apparatuses 106 with serial numbers, CAN addresses, and/or media access control (“MAC”) addresses. This association enables the connectivity apparatus 104 to convert messages received via the network connection into a CAN message for a specific medical device. Further, connected medical devices 502, 504 may use the CAN network to communicate with each other, the docking apparatuses 106a to 106f, and/or the connectivity apparatus 104. Such a configuration enables medical device data to be used for hemodynamic monitoring or managing relayed infusions and other multiple-device dependent treatments.

### Medical Device Hub Power Routing Embodiment

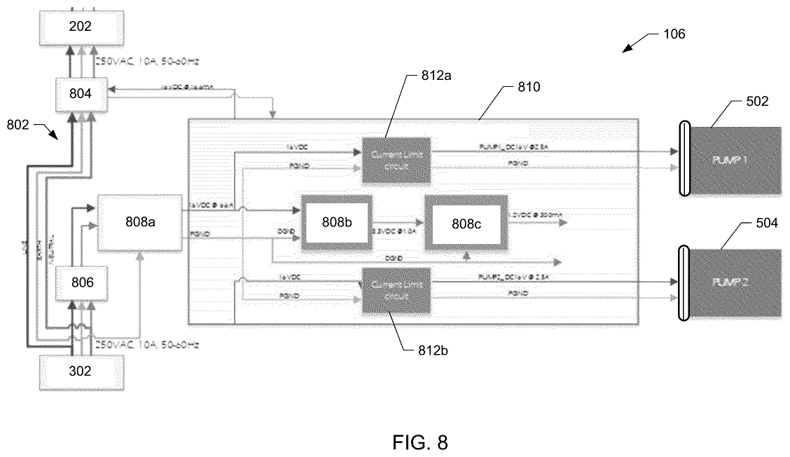

FIG. 8 is a diagram that is illustrative of power routing within the docking apparatus 106 of the medical device hub 100, according to an example embodiment of the present disclosure. As disclosed above, the docking apparatus 106 includes the AC outlet connector 202 positioned at the top of the housing 130 and the AC inlet connector 302 positioned at the bottom of the housing 130. Internally, the housing 130 encloses a power bus 802 that electrically couples the AC inlet connector 302 to the AC outlet connector 202. The power bus 802 includes a line wire, an earth wire, and a neutral wire.

Instead of a direct electrical connection between the connectors 202, 302, the docking apparatus 106 includes a relay switch circuit 804. When another docking apparatus or the connectivity apparatus 104 is connected to the docking apparatus 106 of FIG. 8, the relay switch circuit 804 is configured to be in a closed state, thereby enabling power to reach the AC outlet connector 202. However, when another docking apparatus or the connectivity apparatus 104 is not connected to the docking apparatus 106, the relay switch circuit 804 is configured to be in an open state, thereby preventing power from reaching the AC outlet connector 202.

To power the docking apparatus 106, the power bus 802 is electrically connected to a filter 806. The example filter 806 may include an AC line filter that is configured to suppress electromagnetic interference (“EMI”) on the power bus 802. In some embodiments, the filter 806 may be omitted. An output of the filter 806 is electrically connected to an AC-DC voltage regulator 808a, which is configured to convert the AC voltage from the power bus 802 to a DC voltage. In the illustrated example, the voltage regulator 808a is configured to output 16 volts. In other embodiments, the voltage regulator 808a may output a lower voltage, such as 5 volts or a greater voltage, such as 24 volts.

In the illustrated example of FIG. 8, the 16 volts from the voltage regulator 808a is further down-converted to 3.3 volts by another voltage regulator 808b. Additionally, the 3.3 volts is down-converted to 1.2 volts by yet another voltage regulator 808c. The additional voltage regulators 808b and 808c may be needed if a microcontroller, processor, or circuit of the docking apparatus 106 requires 3.3 volts and/or 1.2 volts to operate.

The docking apparatus 106 may include at least one printed circuit board (“PCB”) 810 that enables at least the voltage regulators 808b and 808c to be mounted thereto. For power routing, the PCB 810 also includes current limiting circuits 812a and 812b that respectively route the output voltage from the voltage regulator 808a to the medical devices 502, 504. The current limiting circuits 812a and 812b are configured to prevent the medical devices 502, 504 from drawing too much power from the voltage regulator 808a, and the power bus 802 generally. As such, the current limiting circuits 812a and 812b provide short-circuit protection in the event of a failure at one of the medical devices 502, 504.

### Docking Apparatus Communication Connections Embodiment

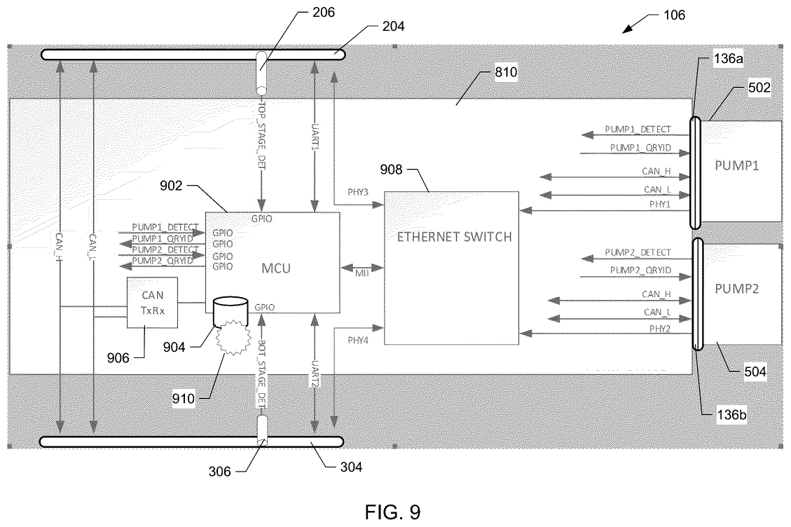

FIG. 9 is a diagram that shows communication connections within the docking apparatus 106 of FIGS. 1 to 6, according to an example embodiment of the present disclosure. In addition to the power circuitry shown in FIG. 8, the docking apparatus 106 of FIG. 9 includes circuitry to facilitate communication between medical devices and the connectivity apparatus 104. In particular, the docking apparatus 106 includes a microcontroller 902 that is communicatively coupled to a memory device 904. The docking apparatus 106 may also include a communication bus transceiver 906 (e.g., a CAN transceiver) and an Ethernet switch 908.

The microcontroller 902 may include a processor, microprocessor, logic circuit, control unit, etc. configured to execute one or more instructions stored in the memory device 904. Execution of those instructions causes the microcontroller 902 to perform the operations discussed herein. The memory device 904 may include any flash memory, ROM, RAM, etc. In addition to storing instructions for the microcontroller 902, the memory device 904 stores local position enumeration data 910. As described herein, the local position enumeration data 910 includes an identifier of the docking apparatus 106, such as a serial number or a MAC address. The local position enumeration data 910 may also include a position value of the docking apparatus 106 within the medical device hub 100. The local position enumeration data 910 may further include at least one base slot position that corresponds to available slots for medical devices on the docking apparatus 106.

As described in connection with FIGS. 2 and 3, the microcontroller 902 is communicatively coupled to the top data connector 204 and the bottom data connector 304. Each of the data connectors 204 and 304 include pins for bus communication, such as CAN communication. The communication bus transceiver 906 is configured as a bus interface for the microcontroller 902. The bus transceiver 906 may be assigned a CAN or other bus address to enable communications to be routed to/from the microcontroller 902 via the bus communication connections. The docking apparatus 106 accordingly contains a portion of the communication bus that is provided between the bus transceiver 906 and the data connectors 204 and 304.

As discussed above in conjunction with FIGS. 2 and 3, the connector 204 includes the detection pin 206 and the connector 304 includes the detection pin 306. The detection pin 206 is communicatively coupled to a first general-purpose input/output (“GPIO”) pin of the microcontroller 902 and the detection pin 306 is communicatively coupled to a second GPIO pin of the microcontroller 902. As discussed above, a voltage on these pins 206 and 306 is pulled low, high, or to ground when another docking apparatus 106 or the connectivity apparatus 104 is connected, thereby indicating the connection to the microcontroller 902.

The connectors 204 and 304 include Ethernet connections, which are routed to the Ethernet switch 908. Each of the microcontroller 902 and/or the medical devices 502 and 504 may include network addresses. The Ethernet switch 908 is configured to route messages to/from the microcontroller 902, the medical devices 502 and 504, other docking apparatuses 106, the connectivity apparatus 104, and/or the gateway server 602. The Ethernet switch 908 may store a routing-and-forwarding table for transmitting messages to/from the docking apparatus 106 and ensuring received messages are transmitted correctly between the microcontroller 902 and the medical devices 502 and 504.

The connectors 204 and 304 also include, in some embodiments, a serial connection. The serial connection may include a UART connection. In other embodiments, the serial connection may include a single-pair Ethernet connection, a Fieldbus connection, a HART connection, etc. The serial connection enables point-to-point communication between the connectivity apparatus 104 and the docking apparatuses 106.

As discussed in connection with FIGS. 1 and 2, the docking apparatus 106 also includes the device connectors 136a and 136b. The docking apparatus 106 includes a device connector 136 for each slot configured to receive a medical device. In this example, the docking apparatus 106 includes the two device connectors 136a and 136b. The device connectors 136 each include pins for an Ethernet connection, which are coupled to the Ethernet switch 908. The device connectors 136 also each include bus (e.g., CAN) connection pins, which are communicatively coupled to the communication bus within the docking apparatus 106. Each of the medical devices 502 and 504 may include bus transceivers for communicating on the communication bus.

Each of the connectors 136 also includes a detection pin that is communicatively coupled to a GPIO pin of the microcontroller 902. Similar to the pins 206 and 306, the detection pin of the device connector 136 is configured to enable the microcontroller 902 to detect when a medical device is connected. The pin may be pulled low, high, or to ground when a medical device is connected to a slot of the docking apparatus 106.

Each of the device connectors 136 also includes a pin for a single-wire device communication connection (e.g., PUMP1_DETECT in FIG. 9) with a GPIO pin of the microcontroller 902. As described in more detail below, the microcontroller 902 is configured to transmit one or more signals via the device communication connection to cause the receiving medical device to transmit its position to the connectivity apparatus 104 via the communication bus. The microcontroller 902 may also use the device communication connection for establishing a CAN and/or Ethernet connection with the connected medical device. Further, the microcontroller 902 is configured to use the device communication connection to directly communicate with the medical device outside of a CAN or Ethernet connection.

The example microcontroller 902 is configured to associate each of the device connectors 136 with a base slot position. For example, the device connector 136a may be associated with a first base slot position within the local position enumeration data 910. Additionally, the device connector 136b may be associated with a second base slot position within the local position enumeration data 910. When a medical device is connected to the respective connector 136, the microcontroller 902 is configured to store an identifier of the medical device in association with the base slot position. For example, the medical device 502 of FIG. 9 is associated with the first base slot position. The detection of the medical device is made by the microcontroller 902 detecting a voltage on the detection pin of the connector 136a. Thus, when a medical device is connected to the docking apparatus 106, the voltage on the detection pin of the connector 136 provides an indication to the microcontroller 902 that the corresponding slot is occupied, which is used by the microcontroller 902 for detecting positions of medical devices.

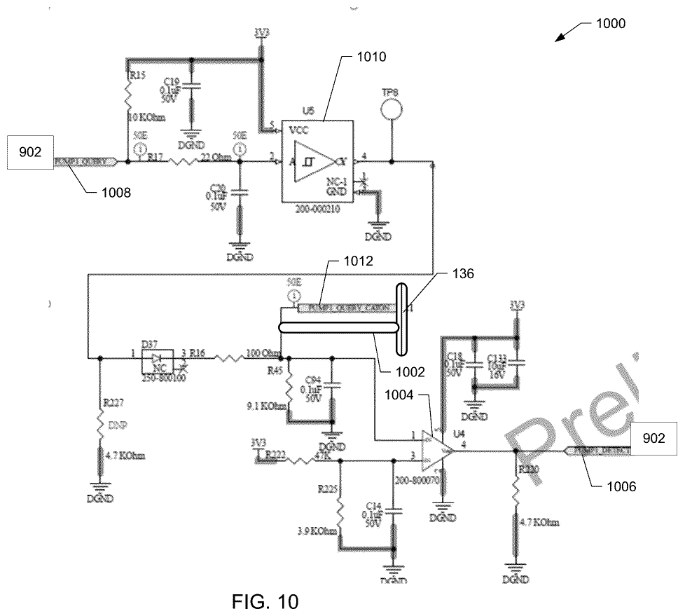

FIG. 10 is a diagram of a circuit 1000 located on the PCB 810 of the docking apparatus 106, according to an example embodiment of the present disclosure. The example circuit 1000 includes a pin 1002 that is located in the connector 136 used for detecting a presence of a medical device. The pin 1002 is electrically coupled to a comparator 1004, which is configured to output a high voltage, such as 3.3 volts when a medical device is connected. In the illustrated example, the comparator 1004 compares a voltage level on the pin 1002 to a reference voltage, such as 3.3 volts. The circuit 1000 is configured to pull the pin 1002 low or to ground. Thus, when a medical device is not connected and the pin 1002 is pulled to ground, the comparator 1004 outputs zero volts, which is received by a GPIO pin 1006 of the microcontroller 902. The example microcontroller 902 is configured to determine that no medical device is connected because the voltage received by the GPIO pin 1006 is zero volts. However, when a medical device is connected, the medical device pulls the voltage on the pin 1002 to, for example, 3.3 volts. The comparator 1004 determines the voltage on the pin 1002 now matches the reference voltage and outputs a positive result, such as 3.3. volts, which is received by the GPIO pin 1006 of the microcontroller 902. The microcontroller 902 uses the 3.3 volts on the GPIO pin 1006 to determine that a medical device is connected.

It should be appreciated that the circuit 1000 shown in FIG. 10 is only one way of detecting a medical device. In other embodiments, different circuits may be used. For example, the detection circuit may instead be pulled low or to ground when a medical device is connected. Alternatively, a buffer circuit may be used to detect a voltage output by a medical device when connected to the docking apparatus 106. Further, while the circuit 1000 is shown for one of the device connectors 136a or 136b, it should be appreciated that the PCB 810 includes an identical circuit for the other of the device connectors.

After a medical device is detected, the microcontroller 902 is enabled to communicate with the medical device. As discussed below, the microcontroller 902 is configured to communicate with the medical device using another GPIO pin 1008 for position detection and establishing bus and/or Ethernet communication. As shown in FIG. 10, the circuit 1000 includes the GPIO pin 1008 of the microcontroller 902, which is provisioned for transmitting a signal that is configured to cause the medical device to transmit its serial number, MAC address, and/or network identifier over the communication bus. The signal may include a serial communication pulse train, such as a pulse width encoded 4-bit number by toggling a voltage provided on the GPIO pin 1008 high and low. The 4-bit number may indicate an overall slot position for the medical device.

The example circuit 1000 includes a buffer 1010 for communicating the pulse train to a query pin 1012 of the connector 136. The buffer 1010 may be configured to delay or queue portions of the pulse train until a processor on a medical device can receive and process the individual voltage transitions. In some instances, the buffer 1010 may be omitted or replaced with logic gates or transistors for communicating the serial signal.

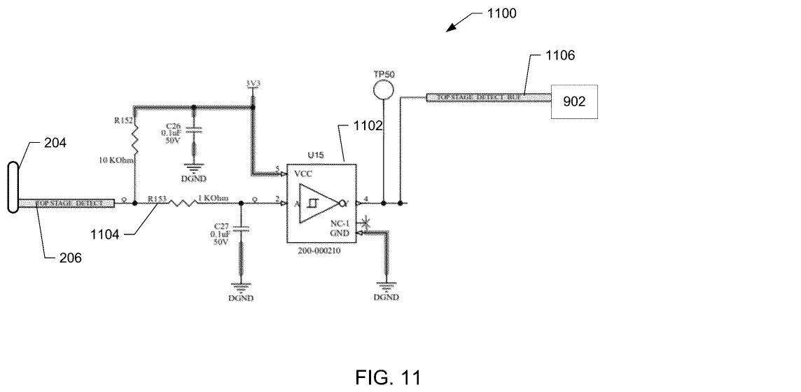

As discussed above, the docking apparatus 106 is configured to detect connection to the connectivity apparatus 104 and/or another docking apparatus 106. FIG. 11 is a diagram of a detection circuit 1100 that is provided on the PCB 810 of FIG. 8, according to an example embodiment of the present disclosure. The example detection circuit 1100 includes an inverter 1102 with a Schmitt trigger, which receives a 3.3 DC voltage from the voltage regulator 808b. The detection circuit 1100 also includes resistors R152, R153 and capacitors C26, C27 that regulate a voltage on an input line 1104 to the inverter 1102. The input line 1104 is electrically connected to the detection pin 206.

When another docking apparatus 106 or the connectivity apparatus 104 is not connected, the inverter 1102 is configured to provide a low signal or voltage to a detection buffer pin 1106 of the microcontroller 902. However, when another docking apparatus 106 or the connectivity apparatus 104 is connected, the pin 206 and the input line 1104 is pulled to ground or a lower voltage as a result of the pin 306 of the bottom data connector 304 being connected to ground or a lower voltage via, for example, a general-purpose input/output (“GPIO”) pin of a microcontroller. When this connection occurs, the inverter 1102 is configured to output a higher voltage to the detection buffer pin 1106 of the microcontroller 902, which indicates the connection of the other, higher docking apparatus 106 or the connectivity apparatus 104.

In some embodiments, the microcontroller 902 is configured to operate an algorithm specified by the instructions for further detecting a connection of the other docking apparatus 106 or the connectivity apparatus 104. The algorithm may specify a time and/or voltage threshold for detecting the connection. For example, the algorithm may specify that the voltage received on the detection buffer pin 1106 of the microcontroller 902 has to be high for at least 500 milliseconds, 1 second, 2 seconds, 5 seconds, etc. before a detection is detected. Additionally or alternatively, another threshold may specify a voltage limit, such as 3.0 volts. Detection of the connection may only occur if the signal output by the inverter 1102 is greater than this voltage limit. A similar voltage and/or timing threshold may be used for the circuit 1000 of FIG. 10 for detecting connection of a medical device.

### Connectivity Apparatus Communication Connections Embodiment

FIG. 12 is a diagram of at least some communication connections provided by the connectivity apparatus 104, according to an example embodiment of the present disclosure. Similar to the docking apparatus 106, the connectivity apparatus 104 includes the AC inlet connector 302 and the bottom data connector 304. The AC inlet connector 302 is configured to receive power from an AC outlet connector 202 of a lower connected docking apparatus 106. Similarly, the bottom data connector 304 is configured to connect to the top data connector 204 of a lower connected docking apparatus 106. As shown, a pin of a microcontroller 1202 of the connectivity apparatus 104 is pulled to ground or provided with a low voltage, which is electrically connected to the pin 306 of the bottom data connector 304. When the connectivity apparatus 104 is connected to a lower docking apparatus 106, the pin 306 connects to pin 206 of the top data connector 204, which causes the input to the inverter 1102 of the detection circuit 1100 to be pulled to the low voltage of ground.

The example microcontroller 1202 is communicatively coupled to a communication bus transceiver 1204, which is communicatively coupled to the data connector 304 via a portion of a communication bus. In the illustrated example, the communication bus transceiver 1204 is configured as a bus interface for the microcontroller 1202. The communication bus transceiver 1204 may be assigned a CAN or other bus address to enable communications to be routed to/from the microcontroller 1202 via the bus communication connections.

The connectivity apparatus 104 may also include an Ethernet switch 1206 for communicating over one or more Ethernet connections within the medical device hub 100 and/or the gateway server 602. Further, the connectivity apparatus 104 may include a USB interface and/or port 1208, for example, for communicatively coupling to the monitoring device 610 via a USB protocol. The connectivity apparatus 104 may also include a UART or other serial communication connection that is provided between the microcontroller 1202 and the data connector 304 to enable point-to-point communication with an adjacent docking apparatus 106. In some embodiments, the connectivity apparatus 104 is configured for Wi-Fi connectivity with the gateway server 602 and/or the monitoring device 610. Further, the connectivity apparatus 104 may connect to the docking apparatus 106 via a Bluetooth® or Zigbee™ connection.

The connectivity apparatus 104 is configured to determine a position of connected medical devices, which is stored as position enumeration data 622. As discussed above in connection with FIG. 6, the position enumeration data 622 is stored in the memory device 620. FIG. 12 shows an example of the position enumeration data 622. It should be appreciated that in other embodiments the position enumeration data 622 may be in different formats.

In the illustrated embodiment, the position enumeration data 622 includes a series of values in conjunction with a medical device serial number. A CAN address, MAC identifier, and/or an IP address may additionally be stored as the position enumeration data 622. Further, an identifier of corresponding docking apparatus 106 may further be stored as the position enumeration data 622.

The sequence of values provides medical device position information that is used for configuring an appearance of the graphical representation 700 of the medical device hub. A first value indicates a stack orientation of the medical device hub 100. A value of ‘0’ indicates the medical device hub 100 has a horizontal orientation while a value of ‘1’ indicates the medical device hub 100 has a vertical orientation. A second value indicates a stack index direction for the medical device hub 100. A value of ‘0’ indicates the ordering of the slot or stack positions of the medical devices are top-down while a value of ‘0’ indicates that ordering is bottom-up. In other words, the overall slot position can be ordered top-down where the top-most medical device within the hub 100 is assigned an overall slot position of ‘1’ or bottom-up where a bottom-most medical device within the hub 100 is assigned the overall slot position of ‘1’.

A third value within the position enumeration data 622 indicates whether a corresponding docking apparatus 106 for a medical device has a vertical orientation (value of ‘1’) or a horizontal orientation (value of ‘0’). A fourth value within the position enumeration data 622 indicates whether the slot assignments for a respecting docking apparatus are top-down or bottom-up. As shown in FIG. 12, the first four values are the same for each medical device. Specifically, the first four values are {1}, {0}, {1}, {0}. This sequence indicates that the medical device hub 100 is in a vertical orientation and the medical devices are indexed top-down. Further, each of the docking apparatuses 106 have a vertical orientation and the medical device slots are indexed top-down.

The next four values within the position enumeration data 622 are specific to a medical device. A fifth value specifies a serial number of the medical device. The fifth value may also specify a serial number or identifier of a corresponding docking apparatus, a CAN identifier of the medical device, and/or a MAC address of the medical device. A sixth value specifies whether the medical device has a horizontal or vertical orientation. In this example, each medical device has a vertical orientation, specified by a value of ‘1’. A seventh value indicates an overall stack or slot position for a medical device. As shown in FIG. 7, the medical devices are sequentially assigned slot position values in a top-down manner such that a top-positioned medical device is assigned an overall slot position of ‘1’ while a next-positioned medical device is assigned an overall slot position of ‘2’. A last or bottom-positioned medical device is assigned an overall slot position of ‘6’.

An eighth value specifies whether the medical device is located in a top or bottom slot of the docking apparatus. The eighth value is based on a base slot position that is determined by the respective docking apparatus 106 based on the correspondence (discussed above in connection with FIG. 9) between the device connectors 136 and assigned base slot positions. In the illustrated example, the first four medical devices are located in a top slot (corresponding to a value of ‘1’) and the bottom two medical devices are located in a bottom slot (corresponding to a value of ‘2’), which is shown in FIG. 7.

In some embodiments, the position enumeration data 622 may include a position number for each of the docking apparatuses 106. The position number indicates which of the medical devices are connected to each of the docking apparatuses 106. The position number may be used instead of storing a serial number for each of the docking apparatuses 106 as part of the position enumeration data 622.

### Medical Device Position Detection Embodiment

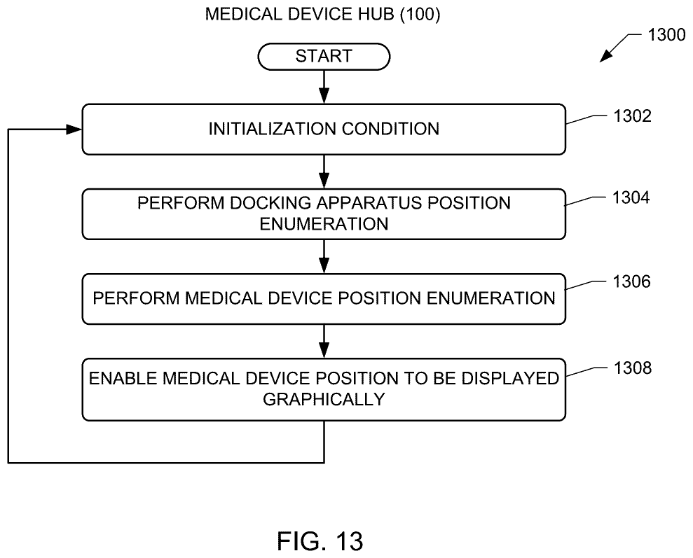

FIG. 13 shows a flow diagram illustrating an example procedure 1300 for detecting positions of medical devices within the medical device hub 100, according to an example embodiment of the present disclosure. The example procedure 1300 may be carried out by, for example, the connectivity apparatus 104 and the docking apparatus 106 described in conjunction with FIGS. 1 to 12. Although the procedure 1300 is described with reference to the flow diagram illustrated in FIG. 13, it should be appreciated that many other methods of performing the functions associated with the procedure 1300 may be used. For example, the order of many of the blocks may be changed, certain blocks may be combined with other blocks, and many of the blocks described are optional.

The procedure 1300 begins when an initialization condition occurs (block 1302). The initialization condition causes the microcontroller 1202 of the connectivity apparatus 104 to determine and/or update positions of medical devices that are connected to the medical device hub 100. The initialization condition may include the microcontroller 1202 detecting or receiving a signal or message indicative that a docking apparatus 106 has been added or removed from the hub 100. In some embodiments, a docking apparatus 106 may detect the direct connection or disconnection of another docking apparatus. In response to the detecting, the microcontroller 902 of the docking apparatus 106 may transmit a UART and/or CAN message to the microcontroller 1202 of the connectivity apparatus 104. Alternatively, the microcontroller 1202 may be configured to use CAN messaging to directly detect when a docking apparatus 106 is connected or disconnected. For example, the lack of a response to a CAN request or ping may be indicative that a docking apparatus 106 has been removed.

The initialization condition may also include detecting when a medical device has been removed or connected to a docking apparatus 106. The microcontroller 1202 may use CAN messaging, for example, to detect when a medical device has been connected or removed. Alternatively, a docking apparatus 106 to which a medical device is attached may transmit a signal or message to the microcontroller 1202 to indicate when the medical device is disconnected or connected.

The initialization condition may also include an indication a treatment is to start, detecting the medical device hub 100 is newly powered on, or detecting the medical device hub 100 has been moved to a new location. The indication of the treatment and movement detection may be received in the microcontroller 1202 from the gateway server 602, the monitoring device 610, and/or one or more connected medical devices. The detection of movement may arise from detecting a new network connection or using an inertial sensor or accelerometer within a medical device to detect movement.

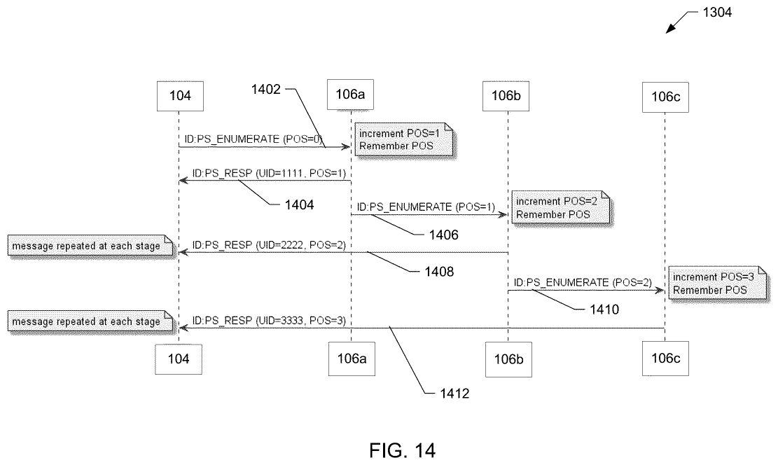

After the initialization condition is detected, the microcontroller 1202 of the connectivity apparatus 104 is configured to perform docking apparatus position enumeration (block 1304). FIG. 14 is a diagram illustrative of performing docking apparatus position enumeration, according to an example embodiment of the present disclosure. To begin, the microcontroller 1202 of the connectivity apparatus 104 is configured to transmit an enumeration request message 1402 to a directly connected docking apparatus 106a using the UART connection. The enumeration request message 1402 includes an initial position value, such as a value of ‘0’. The microcontroller 902 of the docking apparatus 106a receives, via the UART connection, the enumeration request message 1402. In response to receiving the message, the microcontroller 902 increments the initial position value. For example, the microcontroller 902 increments the initial position value to a value of ‘1’. The microcontroller 902 may store the incremented position value of ‘1’ to the memory device 904 as the local position enumeration data 910 so that the docking apparatus 106 has information regarding its position within the medical device hub 100.

The microcontroller 902 next transmits an enumeration response message 1404 via the UART connection to the microcontroller 1202 of the connectivity apparatus 104. The enumeration response message 1404 includes an identifier of the docking apparatus 106, such as a serial number, a CAN address, an IP address, and/or a MAC address. The enumeration response message 1404 also includes the incremented position value. The microcontroller 1202 receives the enumeration response message 1404 and stores the incremented position value in conjunction with the identifier of the docking apparatus 106a as the position enumeration data 622. The microcontroller 902 of the docking apparatus 106 also transmits an enumeration request message 1406 to an adjacent-lower docking apparatus using the UART connection. The enumeration request message 1406 includes the incremented position value.

The microcontroller 902 at the next docking apparatus 106b increments the position value further. For example, the position value is incremented to a value of ‘2’ to indicate the docking apparatus 106b is the second docking apparatus sequentially from the connectivity apparatus 104. Similar to the docking apparatus 106a, the docking apparatus 106b transmits an enumeration response message 1408 with its identifier and further incremented position value. The microcontroller 902 of the docking apparatus 106a receives the message 1408 via the UART connection with the docking apparatus 106b and relays the message 1408 to the microcontroller 1202 via the UART connection with the connectivity apparatus 104. Similarly, the microcontroller 1202 receives the enumeration response message 1408 and stores the further incremented position value in conjunction with the identifier of the docking apparatus 106b as the position enumeration data 622.

In the illustrated example, the microcontroller 902 of the docking apparatus 106a uses the detection pin 306 of the bottom data connector 304 and the corresponding circuit 1100 to detect that the docking apparatus 106b is connected. Based on this detected connection, the microcontroller 902 is configured to transmit the enumeration request message 1406 via the UART connection with the docking apparatus 106b. Similarly, the microcontroller 902 of the docking apparatus 106b detects a connection of another docking apparatus 106c, which causes the microcontroller 902 to transmit an enumeration request message 1410 via a respective UART connection. In a similar manner, a microcontroller 902 of the docking apparatus 106c receives the message 1410, further increments the position value, and transmits an enumeration response message 1412 to the docking apparatus 106b via the UART connections, which is relayed to the microcontroller 1202 of the connectivity apparatus 104 via the docking apparatus 106a. In this example, a docking apparatus is not connected to a bottom of the docking apparatus 106c. The microcontroller 902 of the docking apparatus 106c detects that no further docking apparatus is connected and refrains from sending an enumeration request message.

In this manner, the microcontroller 1202 compiles position enumeration data 622 for the docking apparatuses 106. At this point, the position enumeration data 622 specifies the number of docking apparatuses 106 connected to the medical device hub 100 and their respective identifiers. The microcontroller 1202 is configured to use this information for detecting positions of connected medical devices.

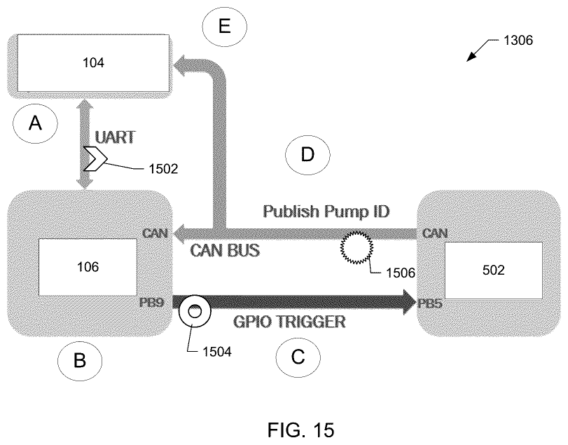

Returning to FIG. 13, after the docking apparatus position enumeration is complete, the microcontroller 1202 of the connectivity apparatus 104 is configured to perform medical device position enumeration (block 1306). FIG. 15 shows a diagram of the medical device position enumeration 1306 performed by the connectivity apparatus 104 in conjunction with the docking apparatuses 106a to 106c, according to an example embodiment of the present disclosure.

The medical device position enumeration begins at Event A when the microcontroller 1202 of the connectivity apparatus 104 transmits a medical device enumeration trigger 1502 over the UART connection with the docking apparatus 106a. The trigger 1502 may include a signal, a message, or a sequence of bits. The microcontroller 902 of the docking apparatus 106a is configured to receive and interpret the trigger 1502 as an indication to start medical device enumeration for connected medical devices. When no medical devices are connected to the docking apparatus 106a, the microcontroller 902 refrains from providing medical device enumeration. Further, when the microcontroller 902 detects another connected docking apparatus, such as the docking apparatus 106b, the microcontroller 902 re-transmits the trigger 1502 via a respective UART connection to the docking apparatus 106b. This re-transmission continues until a bottom-located docking apparatus 106 detects that no further docking apparatuses are connected using a voltage on its respective detection pin 306.

After receiving the medical device enumeration trigger 1502, the microcontroller 902 of the docking apparatus 106 is configured at Event B to determine an overall slot position for each of medical devices that are connected to the docking apparatus 106. To determine the overall slot position, the microcontroller 902 of the docking apparatus 106 uses its assigned position value that was determined during docking apparatus position enumeration described in conjunction with FIG. 14. The microcontroller 902 also uses the base slot positions that correspond to the connectors 136.

In an example, the microcontroller 902 of the docking apparatus 106a determines its position value is ‘1’ from the local position enumeration data 910. The microcontroller 902 then determines which of its connectors 136a and 136b are connected to medical devices based on voltages on respective pins 1006. The local position enumeration data 910 indicates that the connector 136a corresponds to a base slot position of ‘1’ and that connector 136b corresponds to a base slot position of ‘2’. The microcontroller 902 is configured to determine that the overall slot position for the medical device connected to the first (top) device connector 136a is equal to subtracting the base slot position from a result of multiplying the position value by 2 (when there are two medical devices connected to each docking apparatus). Further, the microcontroller 902 is configured to determine that the overall slot position for the medical device connected to the second (bottom) device connector 136b is equal to multiplying the position value by 2 (when there are two medical devices connected to each docking apparatus). Thus, for the docking apparatus 106a, a medical device connected to the device connector 136a has an overall position value of ‘1’ and a medical device connected to the device connector 136b has an overall position value of ‘2’. In another example, for the docking apparatus 106c, a medical device connected to the device connector 136a has an overall position value of ‘5’ and a medical device connected to the device connector 136b has an overall position value of ‘6’. It should be appreciated that the microcontroller 902 is configured to omit determining an overall position value for slots/device connectors 136 that do not have a medical device connected thereto.

After determining the overall position value, at Event C the microcontroller 902 of the docking apparatus 106 selects the appropriate device connector 136 and corresponding medical device and transmits over a device communication connection, such as the GPIO pin 1008 of FIG. 10, a signal 1504. As discussed above, the signal 1504 may include a pulse width encoded 4-bit number that is indicative of the overall slot position.

Responsive to receiving the signal 1504, a medical device 502 at Event D transmits/broadcasts/publishes a message 1506 over the communication bus that includes an identifier of the medical device 502 and the overall slot position, which is received by the microcontroller 1202 of the connectivity apparatus 104. The identifier may include a serial number of the medical device, a CAN address of the medical device, an IP of the medical device, a MAC address of the medical device, etc. In some embodiments, the medical device 502 transmits its serial number and receives a CAN address or identifier from the microcontroller 1202.

At Event E, the microcontroller 1202 of the connectivity apparatus 104 receives the message 1506 and stores the overall slot position as the position enumeration data 622 stored to the memory device. It should be appreciated that the medical device position enumeration 1306 is performed for each of the connected medical devices and each of the docking apparatuses 106 that are connected to the medical device hub 100. At the end of the medical device position enumeration 1306, the connectivity apparatus 104 has an indication of which medical devices are connected to which slots of the docking apparatuses 106 and their respective top-down (or bottom-up) order within the medical device hub 100 via the position enumeration data 622.

It should be appreciated that the position enumeration data 622 may include a sequence of overall position values with missing values corresponding to slots of the docking apparatuses 106 where a medical device is not connected. In some embodiments, the position enumeration data 622 includes the sequence with missing values. In other embodiments, the microcontroller 1202 is configured to re-number the overall position values so there are no missing values. The position enumeration data 622 shown in FIG. 12 includes re-numbered overall position values. If the position values were not re-numbered, for example, the sixth medical device would an over overall position value of ‘12’ instead of ‘6’. At this point, the medical device position enumeration 1306 is complete.

Returning to FIG. 13, the microcontroller 1202 of the connectivity apparatus 104 next enables the graphical representation 700 of the medical device hub 100 to be displayed in one or more user interfaces (block 1308). As discussed above, the clinician device 608, the gateway server 604, and/or the monitoring device 610 may display a user interface that includes the graphical representation 700. The position enumeration data 622 is used to alter the graphical representation 700 to match a number of connected docking apparatuses 106 and medical devices. The graphical representation 700 is used to display medical device data in relation to a graphical representation of a medical device that generated the data. Such a configuration provides an intuitive user interface even when multiple medical devices are simultaneously administering treatments to a patient. The example procedure 1300 re-starts when another initialization condition is detected by the microcontroller 1202 of the connectivity apparatus.

### Conclusion

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should be appreciated that 35 U.S.C. 112(f) or pre-AIA 35 U.S.C 112, paragraph 6 is not intended to be invoked unless the terms “means” or “step” are explicitly recited in the claims. Accordingly, the claims are not meant to be limited to the corresponding structure, material, or actions described in the specification or equivalents thereof.

The invention is claimed as follows:

1. A medical device hub comprising:
a connectivity apparatus including:
a memory device storing a data structure of position enumeration data for docking apparatuses and medical devices,
a microcontroller communicatively coupled to the memory device of the connectivity apparatus,
a first data connector,
a first communication bus transceiver communicatively coupled to the microcontroller,
a first portion of a communication bus communicatively coupled to the first communication bus transceiver and the first data connector, and
a first communication connection provided between the microcontroller and the first data connector; and
a docking apparatus mechanically coupled to the connectivity apparatus, the docking apparatus including:
a memory device storing an identifier of the docking apparatus, a position value of the docking apparatus, and at least one base slot position,
a microcontroller communicatively coupled to the memory device of the docking apparatus,
a second data connector connected to the first data connector of the connectivity apparatus,
a second communication bus transceiver communicatively coupled to the microcontroller of the docking apparatus,
a second portion of the communication bus provided between the second communication bus transceiver and the second data connector,
a second communication connection communicatively coupled to the microcontroller of the docking apparatus and the second data connector,
a device connector for communicatively coupling to a medical device via a device communication connection that is communicatively coupled to the microcontroller of the docking apparatus, the device connector associated with one of the base slot positions, and
a third portion of the communication bus communicatively coupled to the second portion of the communication bus and the device connector,
wherein docking apparatus position enumeration is performed by:
the microcontroller of the connectivity apparatus transmitting, via the first communication connection, an enumeration request message that includes an initial position value,
the microcontroller of the docking apparatus receiving, via the second communication connection, the enumeration request message, incrementing the initial position value, transmitting, via the second communication connection, an enumeration response message that includes the incremented position value and the identifier of the docking apparatus, and storing the incremented position value to the memory device of the docking apparatus, and
the microcontroller of the connectivity apparatus receiving, via the first communication connection, the enumeration response message and storing the incremented position value and the identifier of the docking apparatus to the memory device of the connectivity apparatus as position enumeration data, and
wherein medical device position enumeration is performed by:
the microcontroller of the connectivity apparatus transmitting a medical device enumeration trigger, via the first communication connection,
responsive to the medical device enumeration trigger, the microcontroller of the docking apparatus determining an overall slot position for a medical device connected to the device connector using the incremented position value and the corresponding base slot position,
the microcontroller of the docking apparatus transmitting, via the device communication connection, a signal that is indicative of the overall slot position, causing, the medical device to transmit its medical device identifier and the overall slot position via the communication bus, and
the microcontroller of the connectivity apparatus receiving the medical device identifier and the overall slot position via the communication bus and storing the medical device identifier and the overall slot position to the data structure as position enumeration data.

2. The medical device hub of claim 1, wherein the microcontroller of the docking apparatus transmits the signal to the medical device after detecting that the medical device is connected to the device connector of the docking apparatus.

3. The medical device hub of claim 1, wherein the medical device identifier includes at least one of a serial number, a communication bus address, an IP address, or a media access control (“MAC”) address.

4. The medical device hub of claim 1, wherein the communication bus includes at least one of a controller area network (“CAN”), single-pair Ethernet, a Fieldbus connection, a Highway Addressable Remote Transducer (“HART”) connection, or a serial connection.

5. The medical device hub of claim 1, wherein the docking apparatus position enumeration is additionally performed by:
the microcontroller of the docking apparatus detecting another docking apparatus physically connected to the docking apparatus;
the microcontroller of the docking apparatus transmitting, via a communication connection with the other docking apparatus, a second enumeration request message that includes the incremented position value, causing a microcontroller of the other docking apparatus to further increment the incremented position value and transmit a second enumeration response message that includes the further incremented position value and the identifier of the other docking apparatus;
the microcontroller of the docking apparatus transmitting, via the second communication connection, the second enumeration response message to the connectivity apparatus; and
the microcontroller of the connectivity apparatus receiving, via the first communication connection, the second enumeration response message and storing the further incremented position value and the identifier of the other docking apparatus to the memory device of the connectivity apparatus as additional position enumeration data.

6. The medical device hub of claim 1, wherein the docking apparatus position enumeration and the medical device position enumeration is performed before the medical device performs a treatment.

7. The medical device hub of claim 1, wherein the docking apparatus position enumeration and the medical device position enumeration is performed when the microcontroller of the connectivity apparatus detects the connection of the docking apparatus or detects a connection of a new medical device.

8. The medical device hub of claim 1, wherein the docking apparatus position enumeration and the medical device position enumeration is performed when the connectivity apparatus is powered on.

9. A medical device hub comprising:
a connectivity apparatus including:
a memory device storing a data structure of position enumeration data for docking apparatuses and medical devices, and
a microcontroller communicatively coupled to the memory device of the connectivity apparatus; and
a docking apparatus mechanically coupled to the connectivity apparatus, the docking apparatus including:
a memory device storing an identifier of the docking apparatus, a position value of the docking apparatus, and at least one base slot position,
a microcontroller communicatively coupled to the memory device of the docking apparatus, and
a device connector for communicatively coupling to a medical device via a device communication connection that is communicatively coupled to the microcontroller of the docking apparatus, the device connector associated with one of the base slot positions,
wherein docking apparatus position enumeration is performed by:
the microcontroller of the connectivity apparatus transmitting an enumeration request message that includes an initial position value,
the microcontroller of the docking apparatus receiving the enumeration request message, incrementing the initial position value, transmitting an enumeration response message that includes the incremented position value and the identifier of the docking apparatus, and storing the incremented position value to the memory device of the docking apparatus, and
the microcontroller of the connectivity apparatus receiving the enumeration response message and storing the incremented position value and the identifier of the docking apparatus to the memory device of the connectivity apparatus as position enumeration data, and
wherein medical device position enumeration is performed by:
the microcontroller of the connectivity apparatus transmitting a medical device enumeration trigger,
responsive to the medical device enumeration trigger, the microcontroller of the docking apparatus determining an overall slot position for a medical device connected to the device connector using the incremented position value and the corresponding base slot position,
the microcontroller of the docking apparatus transmitting a signal that is indicative of the overall slot position, causing, the medical device to transmit its medical device identifier and the overall slot position, and
the microcontroller of the connectivity apparatus receiving the medical device identifier and the overall slot position and storing the medical device identifier and the overall slot position to the data structure as position enumeration data.

10. The medical device hub of claim 9, wherein the microcontroller of the docking apparatus transmits the signal to the medical device after detecting that the medical device is connected to the device connector of the docking apparatus.

11. The medical device hub of claim 9, wherein the medical device identifier includes at least one of a serial number, a communication bus address, an IP address, or a media access control (“MAC”) address.

12. The medical device hub of claim 9, wherein the docking apparatus position enumeration is additionally performed by:
the microcontroller of the docking apparatus detecting another docking apparatus physically connected to the docking apparatus;
the microcontroller of the docking apparatus transmitting, via a second device communication connection with the other docking apparatus, a second enumeration request message that includes the incremented position value, causing a microcontroller of the other docking apparatus to further increment the incremented position value and transmit a second enumeration response message that includes the further incremented position value and the identifier of the other docking apparatus;
the microcontroller of the docking apparatus transmitting, via the second device communication connection, the second enumeration response message to the connectivity apparatus; and
the microcontroller of the connectivity apparatus receiving, via the device communication connection, the second enumeration response message and storing the further incremented position value and the identifier of the other docking apparatus to the memory device of the connectivity apparatus as additional position enumeration data.

13. The medical device hub of claim 9, wherein the docking apparatus position enumeration and the medical device position enumeration is performed before the medical device performs a treatment.

14. The medical device hub of claim 9, wherein the docking apparatus position enumeration and the medical device position enumeration is performed when the microcontroller of the connectivity apparatus detects the connection of the docking apparatus or detects a connection of a new medical device.

15. The medical device hub of claim 9, wherein the docking apparatus position enumeration and the medical device position enumeration is performed when the connectivity apparatus is powered on.

16. A medical device hub position enumeration method comprising:
transmitting, via a first communication connection from a microcontroller of a connectivity apparatus, an enumeration request message that includes an initial position value;
receiving, via a second communication connection in the microcontroller of a docking apparatus, the enumeration request message;
incrementing the initial position value;
transmitting, via the second communication connection, an enumeration response message that includes the incremented position value and an identifier of the docking apparatus;
storing the incremented position value to a memory device of the docking apparatus;
the receiving, via the first communication connection in the microcontroller of the connectivity apparatus, the enumeration response message; and
storing the incremented position value and the identifier of the docking apparatus to the memory device of the connectivity apparatus as position enumeration data.

17. The medical device hub position enumeration method of claim 16, further comprising:
transmitting a medical device enumeration trigger, from the microcontroller of the connectivity apparatus via the first communication connection;
responsive to the medical device enumeration trigger, determining, via the microcontroller of the docking apparatus, an overall slot position for a medical device connected to a device connector using the incremented position value and the corresponding base slot position;
transmitting, from the microcontroller of the docking apparatus via the device communication connection, a signal that is indicative of the overall slot position, causing, the medical device to transmit its medical device identifier and the overall slot position via a communication bus;
receiving the medical device identifier and the overall slot position, in the microcontroller of the connectivity apparatus via the communication bus; and
storing the medical device identifier and the overall slot position to a data structure, of position enumeration data for docking apparatuses and medical devices, as position enumeration data.

18. The medical device hub position enumeration method of claim 17, wherein the signal is transmitted to the medical device after detecting that the medical device is connected to the device connector of the docking apparatus.

19. The medical device hub position enumeration method of claim 17, wherein the medical device identifier includes at least one of a serial number, a communication bus address, an IP address, or a media access control (“MAC”) address.

20. The medical device hub position enumeration method of claim 17, wherein the enumeration request message is transmitted:
before the medical device performs a treatment, or
after the microcontroller of the connectivity apparatus detects the connection of the docking apparatus or detects a connection of a new medical device.
